(19) United States
(12) Patent Application Publication
(10) Pub. No.: US 2010/0130945 A1
(43) Pub. Date: May 27, 2010

(54) TREATMENT OF TISSUE VIA APPLICATION OF MAGNETIC FIELD

(76) Inventors: Shlomo Laniado, Tel-Aviv (IL); Eli Nhaissi, Old Westbury, NY (US)

Correspondence Address: MARTIN D. MOYNIHAN d/b/a PRTSI, INC., P.O. BOX 16446, ARLINGTON, VA, 22215, US

(21) Appl. No.: 12/312,287

(22) PCT Filed: Oct. 30, 2007
(86) PCT No.: PCT/IL2007/001325

§ 371 Date: Jan. 21, 2010

Related U.S. Application Data
(60) Provisional application No. 60/856,038, filed on Nov. 2, 2006.
(60) Provisional application No. 60/900,095, filed on Feb. 8, 2007.
(60) Provisional application No. 60/924,305, filed on May 8, 2007.
(60) Provisional application No. 60/935,419, filed on Aug. 10, 2007.

(51) Int. Cl. A61M 35/00; A61N 2/04; A61B 5/0402

(52) U.S. Cl. 604/290; 600/14; 600/509

(57) ABSTRACT

A method of preconditioning a potentially ischemic myocardium or treating a myocardium under an ischemic distress event is disclosed. The method comprises exposing the myocardium to an alternating magnetic field at a frequency of from 15.5N Hz to about 16.5N Hz or from 7.8 Hz to 8.2 Hz and duration selected so as to activate potassium ATP channels in the myocardium, wherein N is a positive integer.

### FIELD AND BACKGROUND OF THE INVENTION

The present invention relates to medical devices and, more particularly, to a device, system and method for treating a tissue by application of magnetic field.

Several studies have been conducted heretofore to investigate the effect of magnetic field on biological material. Representative examples include Bawin et al (1975), Bawin and Adey (1976), Blackman et al (1982, 1985), Schwartz et al (1990, 1993), Sastre et al (1998), Graham and Sastre (2000) and Fitzsimmons et al (1994).

Bawin et al (1975) and Bawin and Adey (1976), found that exposing brain tissue to weak VHF radio signals modulated at 16 Hz released calcium ions bound to the surfaces of the cells. Blackman et al (1982, 1985) testing different field-strengths and frequencies and arrived at the conclusion that the effect of weak fields is oftentimes higher than the effect of strong fields.

Schwartz et al (1990, 1993) demonstrated that the intact myocard of the frog, akin to brain tissue of neonatal chicken, could exhibit movements of calcium ions in response to exposure to weak VHF fields modulated at 16 Hz.

Sastre et al (1998), and Graham and Sastre (2000) exposed the brains of young men overnight to an intermittent magnetic fields (16 Hz, 28.3 microtesla), and found decreases in heart rate in comparison to control. They related their observations to cardiac autonomic control mechanisms and stipulated that such indirect effects were induced by the effect of the magnetic fields on the brain.

Fitzsimmons et al (1994) showed an increase in net calcium flux in bone cells as a result of combined low-amplitude static and alternating magnetic fields, with a peak effect at 15.3-16.3 Hz.

Several theories have been proposed in regard to the biologic mechanism which is responsible to the electrophysiologic response to magnetic fields.

Ledenev (1991) and Blanchard and Blackman (1994) proposed an ionic parametric resonance model (IPR) which assumes field-induced changes in combinations of ions within biologic systems.

Blackman (1982), McLoad and Libof (1986), Smith (1987) and Liboff (1987) proposed an ion cyclotron resonance (ICR) model which assumes that the resonance frequency of calcium ions is 16 Hz and that exposure of cells to a combination of static and 16 Hz alternating magnetic fields causes calcium efflux from the cells. They suggested that the effect of this frequency depended also on the combination with static magnetic fields.

### SUMMARY OF THE INVENTION

Some embodiments of the present invention relate to treatment of various organs by inducing activation of potassium ATP channels. The activation in various exemplary embodiments of the invention is by exposing the organ of interest to an alternating magnetic field at low frequency (typically below 300 Hz).

Other embodiments of the present invention relate to the shielding of various organs so as to prevent or substantially reduce exposure of these organs to low frequency alternating magnetic fields. The shielding is via ferromagnetic materials which can be arranged, e.g., as a closed shape embedded in garment so as to prevent or substantially reduce penetration of the alternating magnetic field therethrough.

According to an aspect of some embodiments of the present invention there is provided a method of preconditioning a potentially ischemic myocardium of a subject, comprising exposing the myocardium to an alternating magnetic field at a frequency and duration selected so as to activate potassium ATP channels in the myocardium.

According to an aspect of some embodiments of the present invention there is provided a method of treating a myocardium under an ischemic distress event of a subject, comprising exposing the myocardium to an alternating magnetic field at a frequency and duration selected so as to activate potassium ATP channels in the myocardium.

According some embodiments of the present invention the ischemic distress event comprises at least one event selected from the group consisting of angina, unstable angina, intermediate coronary syndrome, myocardial infarction, and ischemia due to no-reflow during percutaneous coronary intervention.

According some embodiments of the present invention the method further comprises performing a percutaneous coronary intervention following the exposure to the alternating magnetic field.

According some embodiments of the present invention the exposure to the alternating magnetic field is performed so as to reduce or prevent no-reflow phenomenon in the myocardium.

According some embodiments of the present invention the method further comprises monitoring ECG signals from the subject.

According some embodiments of the present invention the exposure to the alternating magnetic field is at least until a QT interval of characterizing the ECG signal is shortened. by at least 5%.

According to an aspect of some embodiments of the present invention there is provided a method of treating a smooth muscle conduit of a subject, comprising exposing the smooth muscle conduit to an alternating magnetic field at a frequency of and duration selected so as to activate potassium ATP channels in cells of the smooth muscle conduit.

According some embodiments of the present invention the smooth muscle conduit comprises a blood vessel.

According some embodiments of the present invention the smooth muscle conduit comprises a retinal arteriole.

According some embodiments of the present invention the smooth muscle conduit comprises a gastrointestinal tract.

According some embodiments of the present invention the smooth muscle conduit comprises a bladder.

According some embodiments of the present invention the smooth muscle conduit comprises a kidney.

According some embodiments of the present invention the smooth muscle conduit comprises a trachea.

According to an aspect of some embodiments of the present invention there is provided a method enhancing blood perfusion of a dermal layer of a subject, comprising exposing the dermal layer to an alternating magnetic field at a frequency and duration selected so as to activate potassium ATP channels in the dermal layer.

According some embodiments of the present invention the dermal layer is a part of a face of the subject.

According some embodiments of the present invention the dermal layer is a part of a scalp of the subject.

According some embodiments of the present invention the method further comprises administrating to the subject a potassium ATP channel opener drug.

According some embodiments of the present invention the subject is diabetic.

According to an aspect of some embodiments of the present invention there is provided a drug delivery method, comprising administrating to a subject a therapeutic amount of at least one drug and exposing an organ of the subject to an alternating magnetic field at a frequency and duration selected so as to activate potassium ATP channels in the organ.

According to an aspect of some embodiments of the present invention there is provided a method of reducing the likelihood for skin damage to an organ of a subject being irradiated by solar radiation, comprising exposing the organ to an alternating magnetic field while the organ is irradiated by the solar radiation.

According some embodiments of the present invention the exposure to the alternating magnetic field comprises applying the alternating magnetic field to a skin of the subject.

According to an aspect of some embodiments of the present invention there is provided a medical device, comprising a patch designed attachable to a skin of a subject, and an alternating magnetic field generator mounted on or integrated in the patch and configured for generating an alternating magnetic field in the direction of the skin.

According some embodiments of the present invention the patch comprises at least one medicament incorporated therein for intradermal or transdermal delivery of the at least one medicament to the subject.

According to an aspect of some embodiments of the present invention there is provided a mobile system, comprising a cellular telephone unit and an alternating magnetic field generator configured for generating an alternating magnetic field.

According some embodiments of the present invention the alternating magnetic field has a frequency of from 15.5N Hz to about 16.5N Hz.

According some embodiments of the present invention the alternating magnetic field has a frequency of from 7.8 Hz to 8.2 Hz.

According some embodiments of the present invention the alternating magnetic field generator is detachable from the cellular telephone unit.

According some embodiments of the present invention the device or system further comprises a user interface for selecting or adjusting at least one parameter characterizing the alternating magnetic field.

According some embodiments of the present invention the alternating magnetic field is generated at an intensity which is less than 10 microteslas.

According to an aspect of some embodiments of the present invention there is provided a shielding device, comprising a ferromagnetic core having a closed shape and being embedded in a garment designed to be worn by a subject and cover an organ thereof, wherein the ferromagnetic core is designed and constructed to substantially prevent penetration of an alternating magnetic field at a frequency of less than 300 Hz therethrough.

According some embodiments of the present invention the organ is a chest.

According some embodiments of the present invention the organ is a abdomen.

According some embodiments of the present invention the subject is a pregnant woman.

According some embodiments of the present invention the ferromagnetic core comprises a plurality of layers.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-D are schematic illustrations a medical device adapted for treating a heart (FIG. 1A), an eye (FIG. 1B), an abdomen (FIG. 1C) and a foot (FIG. 1D) of a subject, according to various exemplary embodiments of the present invention;

FIGS. 2A-C are schematic illustrations of a mobile system according to various exemplary embodiments of the present invention;

FIGS. 3A-G are schematic illustrations of a magnetic field generator, which in some embodiments of the present invention can be integrated in the system illustrated in FIG. 2A-C, and in other embodiments of the present invention can be used as a separate unit;

FIGS. 4A-D are schematic illustration of a medical device in embodiments of the present invention in which the device comprises an attachable patch;

FIGS. 5A-G are schematic illustration of a shielding device according to various exemplary embodiments of the present invention;

FIGS. 6A-B show examples of a typical ECG changes observed in a rat;

FIGS. 7A-C show changes in Ca2+ transients obtained from isolated cardiac myocytes during magnetic field of 16 Hz;

FIGS. 8A-C show changes in Ca2+ transients obtained from isolated cardiac myocytes with glibenclamide and during magnetic field of 16 Hz;

FIGS. 9A-C show changes in Ca2+ transients obtained from isolated cardiac myocytes with Nicorandil and during magnetic field of 16 Hz;

FIGS. 10A-B show examples of typical ECG changes observed in healthy human volunteer when exposed to a magnetic field according to various exemplary embodiments of the present invention;

FIG. 11 shows an example of a tissue Doppler study, performed for healthy human volunteer exposed to a magnetic field according to various exemplary embodiments of the present invention; and

FIG. 12 shows temperature changes in facial skin following exposure of the skin to magnetic field according to various exemplary embodiments of the present invention.

### DESCRIPTION OF EXEMPLARY EMBODIMENTS

The present embodiments comprise a method, device and system which can be used for medical treatment or for prevention of medical conditions. Specifically, but not exclusively, the present embodiments can be used for many types of treatments, including, without limitation, enhancement of blood perfusion or smooth muscle conduit relaxation, preconditioning of a myocardium, treatment of a myocardium under an ischemic distress event, enhancement of drug delivery, reduction of likelihood for skin damage and the like. Some embodiments of the present invention are useful for shielding body organs against magnetic fields which are typically generated by home appliances.

The principles and operation of a method, device and system according to the present embodiments may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

While conceiving the present invention it has been hypothesized and while reducing the present invention to practice it has been realized that magnetic fields can be used to activate potassium ATP (K_{ATP}) channels. It was found by the present Inventors that K_{ATP} activation in cells' membrane can be achieved by exposing the cell to an alternating magnetic field having a frequency of about 8 Hz or about 16 Hz or integer multiplication (higher harmonic) thereof.

As used herein “about” refers to ±15%.

For the purpose of better understanding of the present invention, following is a description of the role of sarcolemmal potassium ATP channels in tissue.

ATP-sensitive potassium channel is an octomeric protein composed of two dissimilar subunits types, known as a pore forming subunit (Kir 6.2) and the sulfonylurea receptor (SUR), which is the regulatory subunit (SUR2A in the heart and, SUR2B in the smooth muscles residing in the vascular system and in other conduits, e.g., the bronchi or ureter). The co-expression of both these subunits recapitulates the essential properties of the channels.

The channel activity occurs in bursts of brief openings that swiftly alternate with briefer closings. The short active bursting episodes are separated by long-lived inactive interburst intervals.

The gate governing the intraburst kinetics lies close to the selectivity filter. This gate appears to be able to operate independently of the gate regulating the long inter-burst closings determined by the SUR receptors.

Kinetically, and apparently structurally, gating is a multi-component dynamic process. Transitions between the short bursts and the long-closed states can be governed by a “slow” gate that lies at the intracellular mouth of Kir6.2. An additional “fast” gate governs the rapid openings and closings occurring within the short bursts of opening separated by the long-closed states.

Under normal cellular ATP levels, these membrane channels are not significantly active. The probability of K_{ATP} channels to open is normally very low, corresponding to opening of about 3% of the membrane channel population. However, the correlation of this is the potential for a large increase in potassium ion conductance and efflux, if substantial number of clusters of K_{ATP} channels are opened in response to metabolic insult, or other internal or external stimuli.

Due to the high density of K_{ATP} channels in the myocytes, when activated in conditions of metabolic distress such as ischemia, the cellular membrane becomes hyperpolarized, the action potential is significantly shortened with a restriction in its amplitude and the myocyte can become practically unexcitable.

While conceiving the present invention it was hypothesized that an alternating magnetic field can activate dormant K_{ATP} channels via an effect of “periodic forcing” caused through a mechanism known as stochastic resonance (SR).

SR is a nonlinear phenomenon in which the addition of noise to a system increases its response to external signal. SR can allow biologic cells to detect and respond to external signals even when the signals are very weak, e.g., below the thermal noise limit. SR is a statistical process realized in nonlinear systems whereby the noise internal to the system enhances the transmission of weak external information through the system. In SR, the signal to noise ratio at the system's output reaches local maxima when the external signals are applied at frequencies which are integer multiplication of a certain frequency also known as the resonance frequency of the system.

Several studies have been performed in to investigate the SR theory on biological system. (to this end see, e.g., Gammaitoni et al, 1998, Astumian 1997, Menconi et al, 1998, Petracchi et al, 1994, Menconi 1998, Tsong 1992, Weaver and Astumian 1990, and Kruglikov and Dertinger 1993).

The effect of periodic SR is a combination of a source of subthreshold background noise and an external periodic signal at about the resonance frequency or integer multiplication thereof. It was found by the present Inventors that in the case of K_{ATP} channels, the source is enacted by random channel fluctuations switching between open and closed states, and the external periodic signal is enacted by an alternating magnetic field. The external signal activates the K_{ATP} channels residing at the membranes of excitable cells.

The cellular membrane of excitable cells is positively charged relative to the interior of the cells, to effect a so called “resting potential” which is typically about −70 mV for myocytes or neurons. The resting potential of the cells is mainly governed by the level of potassium ions. Upon activation of potassium channels, electrical currents traversing through the open channels make the interior of the cell more negative compared to its normal level, hence induce hyperpolarization of the cellular membrane to effect efflux of intracellular potassium and calcium ions from the myocytes. This phenomenon is promoted by the closure of the membrane L-type voltage-gated channels following the rapid shift of potassium ions through the opened K_{ATP} channels and the hyperpolarization of the membrane.

The motion of the gating charge in a multiple barrier energy landscape, under the influence of thermal noise and a periodic external force, may promote the SR mechanism. The present Inventors have uncovered that ion channels are activated, even when dormant, when exposed to alternating magnetic field at a frequency which approximately matches, or which is approximately an integer multiplication of their open-close frequency. The selected frequency allows overcoming the threshold barrier even when the applied alternating magnetic field is substantially below the thermal threshold of the K_{ATP} channels.

K_{ATP} channels have been reported to have open burst periods of about 3.28 ms separated by interburst intervals of about 59.35 ms [Aguilar-Bryan et al., 1998]. This corresponds to a total switching time of about 62.3 ms, which can equivalently be expressed as a closed-open frequency of about 16 Hz. Similar data were obtained in experiments dedicated to K_{ATP} SUR2 channels, with average open burst periods of 3.3 ms and average interburst intervals of about 62.7 ms, corresponding to an closed-open frequency of about 15.2 Hz.

Revealing the above closed-open frequencies, it was hypnotized by the present Inventors that K_{ATP} channels such as Kir 6.2/SUR 2A and SUR 2B, which are generally dormant under normal conditions can be activated by exposing the cells of interest to an alternating magnetic field at a frequency of from about 15.5N Hz to about 16.5N Hz, where N is a positive integer, preferably lower than 5, more preferably lower than 4, more preferably lower that 3, more preferably N=1. In various exemplary embodiments of the invention the alternating magnetic field has a frequency of 16 Hz. Also contemplated, is a magnetic field at a frequency of from about 7.8 Hz to about 8.2 Hz, e.g., 8 Hz.

The use of the above frequencies ensures matching between the K_{ATP} channels rhythm and the applied alternating magnetic field. Such matching provides the resonance condition to the periodic kinetics of the K_{ATP} channels (as manifested by the frequency of the open-close cycle).

Preferably, the alternating magnetic field of the present embodiments is sufficiently low so as not to ionize or substantially heat the treated tissue. Thus, in various exemplary embodiments of the invention the activation of K_{ATP} channels is promoted by resonance.

The frequencies employed by the present embodiments can activate both SUR2BA and SUR2B regulatory subunits and are therefore suitable for the treatment of myocardial tissue which includes the SUR2A subunit, as well as smooth muscle cell tissue which includes the SUR2B subunit. Thus, the activation of K_{ATP} channels in accordance with some embodiments of the present invention is in the myocytes of various organs and conduits including, without limitation, myocardium, brain neurons, smooth muscle cells inhabiting microvascular systems such as, but not limited to, arterioles and pre-capillaries in various tissues (e.g., skin, brain, kidney, bronchial-airways, retina, etc.), and various conduits having walls inhabited by smooth muscle cells.

As further detailed in the Examples section that follows, experiments performed by the Inventors of the present invention demonstrate the benefits rendered to ischemic tissues by the increase in blood supply due to arteriolar dilation, inducing dual favorable effects on the myocardium when it suffers from metabolic distress such as ischemia and anoxia, via the exposure to the alternating magnetic fields of the present embodiments. Such dual effects can therefore be exploited in accordance with some embodiments of the present invention to protect the heart, e.g., from the detrimental effects of evolving ischemia, by reducing the workload of the myocardium while increasing tissue blood perfusion.

As stated, the activation of K_{ATP} channels residing at the membranes of the excitable cells effects an efflux of calcium ions from the myocytes. In turn, depletion of calcium ions from the myocytes of the myocardium or from the smooth muscle cells results in relaxation of myofibrils, and the reduction in their contractions induces relaxation and vasodilatation of the microvascular system residing in most tissues or other conduits (e.g., second degree airways).

Channel opening precipitates hyperpolarization of the cellular membrane and enhances shortening of phase 3 repolarization of the myocyte action potential thus reducing action potential duration (APD), which in the event of myocardial myocytes, (for example, as experimented by the present Inventors in intact rats), can be expressed in the external ECG, as a QT segment shortening. The enhanced shortening of phase 3 repolarization in addition to membrane hyperpolarization reduces the total amount of Ca2⁺ ions entering into the cell via L-type channels hence prevents calcium overload, favoring myocyte relaxation.

It was found by the inventors of the present invention that the typical elapsed time from the initiation of the exposure to the alternating magnetic field of the present embodiments to the appearance of tissue reaction is from about 4 minutes to about 10 minutes. This is the approximated evolution time scale of a biologic reaction. It was surprisingly uncovered that the recovery of the tissue following termination of the magnetic field was characterized by a considerably longer duration. For example, in experiments performed by the present Inventors on live rats and humans as well as isolated myocardial cells in culture, a period of 30-60 minutes has elapsed until the amplitude of the QRS complex had fully recovered.

Such phenomenon mimics the acute “memory” of ischemic preconditioning, in a similar manner to the cardioprotection produced as a result of isoflurane administration.

Thus, the alternating magnetic field of the present embodiments can provide sustained protection to the myocardium following a preceding ischemic episode.

As further detailed hereinunder, the delayed recovery of K_{ATP} channels following the exposure to the alternating magnetic field of the present embodiments can be exploited in various procedures, including, without limitation, percutaneous coronary intervention and the like.

When the alternating magnetic field of the present embodiments is applied to the heart tissue, the calcium efflux can, for example, alleviate the myocardium in the event of exaggerated workload, so as to enhance its survival in states of ischemia. Thus, the present embodiments are useful for treating ischemic heart. For example, the alternating magnetic field of the present embodiments can be applied to a myocardium under an ischemic distress event, such as, but not limited to, angina, unstable angina, intermediate coronary syndrome, myocardial infarction, and ischemia due to no-reflow during percutaneous coronary intervention.

Depletion of intracellular calcium ions favors energy sparing through reduction in mechanical contraction, ameliorates myocyte workload and favors cellular integrity. The present embodiments can be used in the events of myocardial metabolic distress (such as cardiac ischemic episodes giving rise to anginal pains, or infarction) or conditions that precede cardiac surgery or percutaneous coronary intervention (PCI). The present embodiments can be used for preconditioning a potentially ischemic myocardium.

Ischemic myocardial preconditioning (IPC) is a phenomenon whereby brief periods of ischemia have been shown to protect the myocard against a more sustained and substantial ischemic insult. The result of IPC may manifest eventually in a marked reduction in an evolving myocardial infarction size. This phenomenon has also marked clinical significance in the events of cardiac surgery, intermediate coronary syndrome and other acute ischemic episodes such as unstable angina that often precede the evolving process of myocardial infarction.

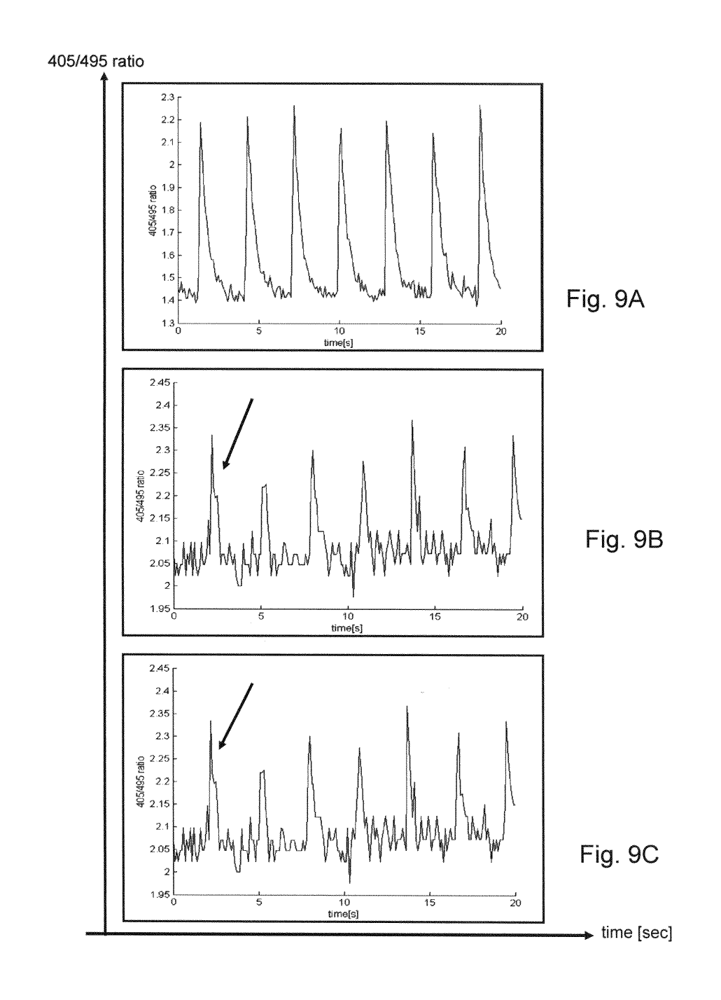

IPC is a phenomenon whereby exposure of the myocardium to a brief episode of ischemia and reperfusion markedly reduces tissue necrosis induced by a subsequent prolonged ischemia. The phenomenon is related to opening of K_{ATP} channels (see, e.g., Cohen et al, 2000, Garrett & Pearle, 2003, and Broadhead et al, 2004) and can therefore be induced using the alternating magnetic field of the present embodiments. In various exemplary embodiments of the invention the application of the alternating magnetic field with the appropriate frequencies is supplemented by administration of one or more pharmaceutical agents, such as, but not limited to, nicorandil. Nicorandil belongs to a group known as potassium channel openers abbreviated KCO. It was found by the Inventors of the present embodiments that the combination of magnetic treatment via exposure of the myocardium to the alternating magnetic field of the present embodiments and pharmacological treatment via administration of e.g., nicorandil, has a synergistic effect in that (i) the alternating magnetic field of the present embodiments facilitates better delivery of the KCO via vasodilatation, and (ii) both the KCO and the magnetic field activate potassium ATP channel. Such synergistic effect is demonstrated in the Examples section that follows, see, e.g., FIGS. 9A-C for the case of 15.95 Hz. the use of KCOs other than nicorandil (e.g., pinacidil and diazoxil) is not excluded from the scope of the present invention.

Kir6.2/SUR2A channels that reside in the membrane of cardiac myocytes and SUR2B subunits inhabiting the vascular smooth muscle cells, belong to the family of K+ internal rectifiers. As such they are affected, in addition to their response to variations in intracellular ATP levels, by voltage variations across the membrane that are determined by other K+ internal rectifies. In general, the K+ internal-rectifiers channels help maintaining the diastolic steady-state changes occurring in the potassium electromotive driving force across the cellular membrane (Wellner-Kienitz et al, 2004).

As stated, the present embodiments can be used in conditions that precede PCI. PCI has become one of the commonly used procedures for treating stenotic coronary artery disease and has been evolved substantially over the past three decades.

Yet, PCI has post procedural elevations in peripheral blood of biochemical markers attesting for peri-procedural myocardial injury, particularly Troponin-T (cTnT). The post-procedural adverse reaction of no-reflow state, frequently accompanied by elevations in myocardial Troponin blood levels as compared with pre-procedural levels, reflects minor or major myocardial damage. Isolated elevation in Troponin-T following PCI was found to be associated with higher long-term morbidity and mortality, and particularly when the PCI involved multivessel procedures. Angiographic no-reflow is associated with an increase in the occurrence of myocardial infarction, heart failure and death. There is frequently a persistent microcirculatory impairment and abnormal tissue level perfusion despite normal epicardial flow. Further details are found in Herrmann (2005), Balin et al (2006), Prasad et al (2006), Nienhuis et al (2007) and a review by Elckhout et al (2001).

Conventional PCI are typically supplemented by periprocedural treatment of vasovasodilators such as verapamil and adenosine for reducing microvascular spasm and the resulting no-reflow responsible for abnormal tissue perfusion following PCI (see, e.g., Michaels et al, 2002, Hang et al, 2005, Vijasa, 2006 and Harding, in an editorial, 2006). However, although early administration of intracoronary verapamil (which is a known Ca2⁺ blocker and vasovasodilator) improves myocardial perfusion during PCI for acute myocardial infarction, in 18% of the cases such treatment is associated with hypotension and complete heart block lasting up to three hours.

The exposure of the myocardium to the alternating magnetic field of the present embodiments prior to non-emergency elective PCI, can condition the heart to have extra protection in a mode similar to that induced by ischemic preconditioning, and can therefore reduce microvascular spasm and the resulting no-reflow phenomenon both in non-emergency and emergency states.

It was found by the Inventors of the present invention that while verapamil blocks L-type voltage-gated Ca2+ channels in the sarcolemma directly, the exposure of the myocardium to the alternating magnetic field of the present invention induces the blocking effect on the calcium channels by indirect effect of activation of K_{ATP} channels, and thus can have no detrimental effects on induction of any heart block or systemic hypotension.

In addition, it was found by the Inventors of the present invention that while administration of verapamil oftentimes has detrimental myocardial or systemic effects, the effects induced by the alternating magnetic field of the present embodiments are substantially localized. This is because the magnetic field effect is limited to the volume at which the field is applied. When the alternating magnetic field of the present embodiments is applied to the heart, the effects induced thereby are localized to the heart substantially without causing any detrimental myocardial or systemic effects.

Most organ conduits in the body are equipped with constricting and dilating systems affected by the modulation of smooth muscle cells in their walls. Such conduits are dedicated to the transfer of necessary supply of blood flow or air, or to the evacuation of biologic detrimental excretion, such as urine. The tone prevailing the flow in these conduits depends on the contractile activity of the smooth muscle cells in their walls.

When the alternating magnetic field of the present embodiments is applied to a smooth muscle conduit, the calcium efflux can relax the smooth muscle cells of the conduit. Thus, the present embodiments are useful for treating a smooth muscle conduit. In the case of smooth muscle cells of the arteriolar system (e.g., cardiac second degree coronary vessels and/or microvascular systems) the present embodiments can be used to induce vasodilation. Representative examples of tissues or organs which can be treated by the alternating magnetic field of the present embodiments include, without limitation, brain, retina and skin.

It was found by the present Inventors that the alternating magnetic field of the present embodiments can also be applied to other and diverse muscle cell types, such as, but not limited to, smooth muscle cells inhabiting the walls of the arterioles, sub-arterioles and pre-capillaries, like those inhabiting the brain, retina, skin etc. This is because in smooth muscle cells, myocyte contractility and vascular constriction or dilation is linked to membrane ion channel activity. For example, influx of calcium ions into the smooth muscle cells through the L-type voltage-gated calcium channels, affects the myocyte myofibrils which are the contracting elements within the smooth muscle cells residing in the vascular walls. Thus, in various exemplary embodiments of the invention the alternating magnetic field of the present embodiments is applied so as to activate and open K_{ATP} channels residing in the membranes of the myocytes inhabiting the vascular system. The alternating magnetic field is preferably applied so as to induce smooth muscle relaxation and microvascular dilation. According to a preferred embodiment of the present invention the alternating magnetic field is used for enhancing blood perfusion to the tissue which is exposed thereto.

In the brain, for example, active K_{ATP} channels are present in the neurons as well as large cerebral arteries and arterioles (see, e.g., Taguchi et al, 1994, Kitazono et al, 1995, Yamada et al, 2001, Sun et al, 2006, Jenorow et al, 1998, Rosenblum 2003, Louis et al, 1996, Amstead, 1996, Faraci, 1993, Faraci and Sobey, 1998, and Santa et al, 2003).

The activation of these channels in accordance with some embodiments of the present invention can induce cerebral vasodilation and increase cerebral blood flow. This is particularly useful in the event of evolving ischemic stroke (CVA). As demonstrated in the Examples section that follows, a blocking effect induced by glibenclamide confirms the activation of Kir6.2/Sur2 (both A and B) channels by the alternating magnetic field of the present embodiments.

In the ureter, application of the alternating magnetic field of the present embodiments can activate K_{ATP} channels inhabiting the walls of the ureter and thus release the ureter from spasms, and alleviate the severe pains of renal colics in patients suffering from renal stones.

In the respiratory airways, application of the alternating magnetic field of the present embodiments can activate K_{ATP} channels inhabiting the walls of the second degree respiratory airways, so as to relax bronchial spasm in asthma. Thus, in various exemplary embodiments of the invention the alternating magnetic field is used for the treatment of asthma.

In the intestinal system, application of the alternating magnetic field of the present embodiments can activate K_{ATP} channels inhabiting the intestinal walls so as alleviate acute GI problems, such as abdominal cramps resulting from partial ileus or severe gastroenteritis.

The present embodiments, as stated, are also useful for treating the retina. The retinal circulation lacks autonomic innervations. The physiologic modulation of retinal vascular tone depends on local control mechanisms, such as metabolic regulation. The predominant site responsible for local blood flow regulation is the microvascular network, particularly in the arteriolar and subarteriolar retinal bed. The retinal tissue has one of the highest metabolic rate in the body with high retinal oxygen demand even under normal conditions. The pathophysiology of diabetic retinopathy is directly related to microvascular reduction in blood flow. It is recognized that mild microvascular insufficiency in the retina may lead to local ischemia and promote diabetic macular edema and, in sever cases, blindness.

When the alternating magnetic field of the present embodiments is applied to the retina, it can activate K_{ATP} channels in the retinal vasculature so as to induce vasodilation. It was conceived by the present Inventors that such treatment can counteract and delay the progression of the detrimental effects of microvascular damage inflicted upon the retina by particularly chronic diabetes and hypertension.

The present embodiments are also useful for enhancing blood perfusion in other diabetic organs such as, but not limited to, limbs. As demonstrated in the Examples section that follows, the exposure of one diabetic foot to the alternating magnetic field of the present embodiments results in a newly acquired or enhanced superficial arterial pulses in both feet. This bilateral effect results through a mechanism of entrainment promoted via the autonomous nervous system responsible for creating near equal effects in both feet. Besides providing the skin of the treated organ with enhanced blood supply, the present embodiments also facilitate intra- or transdermal delivery of drugs (e.g., antibiotic, antiseptic or similar drugs in various formulations such as creams) into the tissue of the skin. The alternating magnetic field of the present embodiments facilitates penetration of the drug into the skin and may also be used for modulating the release rate of slow release agent, because the enhancement of dermal blood perfusion favors medical or cosmetic agent absorption. Thus the present embodiments successfully counteract the deleterious progression of chronic peripheral diabetic pathology and neuropathy related to microvascular deficiencies.

The present embodiments are also useful for enhancing blood perfusion in the skin of other organs, such as, but not limited to, facial skin. It was found by the Inventors of the present invention that the application of the alternating magnetic field of the present embodiments to the facial skin can activate K_{ATP} channels in skin microvascular system so as to enhance blood perfusion thereby to nourish the dermal layer and to improve the viability and quality the skin. The enhanced blood perfusion can also facilitate intradermal or transdermal delivery of agents (e.g., cosmetic agents in various formulations such as creams). The effect of the alternating magnetic field of the present embodiments is similar to that induced by the administration of KCOs for directly activating K_{ATP} channels in keratinocytes (HaCat cells). It was conceived by the present Inventor that the exposure of the skin to the alternating magnetic field of the present embodiments can protect the skin from the deleterious effects induced by solar radiation (particularly, but not exclusively, ultraviolet radiation) and prevent or at least reduce or delay premature aging of the skin, and the emergence of skin tumors (e.g., melanoma and basal-cell-carcinoma).

The appealing complexion of an individual is, to a great degree, dependent on the healthy and youthful looks of his or her facial skin. Among the multiple factors affecting skin appearance, are favorable genetic traits, and avoiding exposure to sun-light, particularly to ultra-violet waves. Appropriate nutrition and external means such as creams, lotions and massages are among the methods commonly used to improve the looks of facial skin. Among the genetic and intrinsic factors responsible for rendering an individual a radiant and appealing complexion, is to a great degree, the optimal amount of blood supplied to facial skin.

The blood vessels responsible for the perfusion of facial skin are minute vessels, arterioles and pre-capillaries that are embedded within the dermal layer of the skin.

Restriction of the amount of blood provided to the skin as a result of arteriolar vasoconstriction may deplete the tissue from oxygen and other essential nutrients. Such unbecoming effects can occur in habitual smokers, in whom the texture of facial skin deteriorates, to exhibit features of premature aging, wrinkles and sallow pale-grey complexion.

The adverse effect of the inhaled nicotine on the skin is mostly a result of arteriolar vasoconstriction. The muscle cells at the walls of the arteriolar vessels—the myocytes, which are smooth muscle cells, surround the lumen of the arteriole, and determine by their contraction, or relaxation, the constriction or dilation of the nutrient vessels. The function of the myocytes, to contract or dilate is dependent on the activation or inactivation of the calcium ion channels inhabiting their cellular membrane. This process is usually dependent on physiological stimuli, such as those coming from the autonomous nervous system, but can also be effected by external stimuli, induced by drugs or different physical agents.

It was found by the present Inventors that enhancement of blood supply to the skin, via arteriolar vasodilation has a beneficial effect on the skin, and in addition to providing some additional appealing blush to the cheeks. Enhancement of blood supply can also help healing skin cut or wounds by supplying increased amount of blood to the marginal regions of the cut or wound.

The primary target of most transdermal or intradermal drug delivery system is to achieve the beneficial effects on the skin through optimal diffusion of medical or cosmetic agents. This is true when the compounds are applied for inducing local topical effects either at the direct level of the skin, or at certain tissues residing beneath it and being closely located to the existing skin lesions.

The therapeutic effects of topically applied drugs in the form of creams depend on variety of factors, including the rate of absorption and the amount and depth into which the drugs penetrate the skin. The present Inventors found that the exposure of the skin to the alternating magnetic field of the present embodiments aids in the healing of various types of tissue damages because the alternating magnetic field effects delivery of increased amount of oxygen and nutrients to the tissue (via enhancement of blood perfusion), and assists in the delivery of intradermal or transdermal agents. The delivery of intradermal or transdermal agents of any type (therapeutic and cosmetic) to the tissue of the skin is augmented by temperature. This is because warming increases the kinetic energy involved in the absorption process through potential pathways. While reducing the present invention to practice it was unexpectedly uncovered that the exposure of the skin to the alternating magnetic field of the present embodiments results in a temperature increment such that the absorption rate of the intradermal or transdermal agents is increased.

The present embodiments are further useful for treating chronic skin wounds, lesions and ulcers which are typically developed due to microvascular ischemia, e.g., in diabetic patients, habitual smoking patients and the like. Smoking subjects oftentimes suffer from vasoconstricting effect in the facial skin caused from inhaled nicotine. The vasoconstricting effect induces a sallow grey-yellowish look to the face and, for some subjects, is pronounced almost immediately following the smoking of a single cigarette. The exposure of the facial skin to the alternating magnetic field of the present embodiments can efficiently counteract the vasoconstricting effect. Thus, in various exemplary embodiments of the invention the facial skin is exposed to the alternating magnetic field following or during smoking.

More generally, any organ damaged from skin wounds, lesions and/or ulcers (e.g., the foot of a diabetic patient, or the face of a smoking patient) can be exposed to the alternating magnetic field of the present embodiments so as to enhance local arterial or arteriolar blood perfusion as further detailed hereinabove.

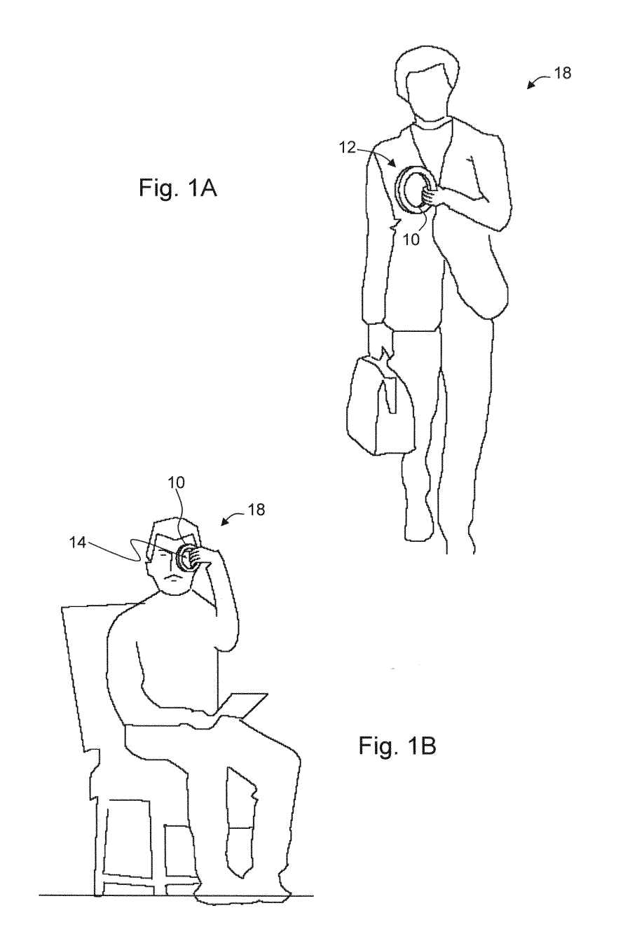

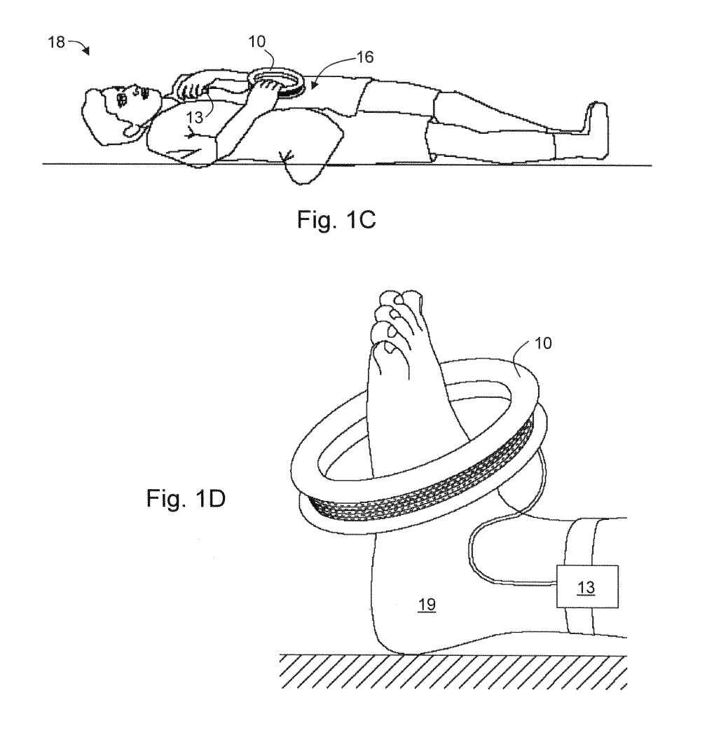

Referring now to the drawings, FIGS. 1A-D illustrate a medical device 10 adapted for treating a heart 12 (FIG. 1A), an eye 14 (FIG. 1B), an abdomen 16 (FIG. 1C) and a foot 19 (FIG. 1D) of a subject 18, according to various exemplary embodiments of the present invention. Device 10 is preferably designed and constituted to provide an alternating magnetic field at a frequency of from about 15.5N Hz to about 16.5N Hz, where N is a positive integer, preferably lower than 5, more preferably lower than 4, more preferably lower that 3, more preferably N=1. In various exemplary embodiments of the invention the alternating magnetic field has a frequency of 16 Hz. Also contemplated, is an alternating magnetic field at a frequency of from about 7.8 Hz to about 8.2 Hz.

The wave shape controlling the alternating magnetic field is preferably sinusoidal but other shapes, such as, but not limited to, square wave, sawtooth wave, triangle wave and the like are not excluded from the scope of the present invention. Device 10 can comprise one or more coils of conducting wire and a power source constructed to generate a current in the coils sufficient to generate the alternating magnetic field in the volume of interest.

The magnitude of the alternating magnetic field generated by device 10 can vary, depending on the organ for which device 10 is adapted. Generally, for an organ which is close to device 10 the magnitude can be lower than for organ which is farther from device 10. This is because the alternating magnetic field decays away from device 10. For example, when device 10 is used for applying alternating magnetic field to the chest, it can decay from an intensity of 4-7 μT at skin level to about 300 nT on the heart. Typically, the RMS intensity of the alternating magnetic field is from several tens of nanoteslas to a few tens of microteslas, e.g., from about 50 nT to about 50 μT, more preferably from about 60 nT to about 10 μT, more preferably from about 100 nT to about 3 μT, more preferably from about 200 nT to about 1 μT, e.g., an RMS intensity of about 300 nT.

It was found by the inventors of the present invention that such intensities are sufficient to activate the potassium channels as further detailed hereinabove.

Thus, when device 10 is held near the chest of the subject, the alternating magnetic field penetrates the thorax wall and activates K_{ATP} channels in the myocardium as further detailed hereinabove. In this embodiment, suitable filed intensities at skin level are, without limitation, from about 4 μT to about 10 μT, but other intensities are not excluded from the scope of the present invention.

When device 10 is held near the eye of the subject, the retinal vasculature (not shown) is exposed to the alternating magnetic field and vasodilation is induced therein. In this embodiment, suitable filed intensities face level are, without limitation, from about 5 nT to about 60 nT, but other intensities are not excluded from the scope of the present invention.

When the device 10 is held near the abdomen of the subject, the alternating magnetic field penetrates the abdomen wall and activates K_{ATP} channels in the GI tract as described above. In this embodiment, suitable filed intensities at skin level are, without limitation, from about 10 μT to about 30 μT, but other intensities are not excluded from the scope of the present invention.

When device 10 is held or mounted on the foot, the skin of the foot is exposed to the alternating magnetic field which activates K_{ATP} channels inhabiting the myocytes of the arterioles in the foot's skin, thereby promoting an increase in dermal arteriolar blood perfusion as further detailed hereinabove. In this embodiment, suitable filed intensities at skin level are, without limitation, from about 4 μT to about 15 μT, but other intensities are not excluded from the scope of the present invention.

When device 10 is held near the face (e.g., the cheek), the facial skin of the foot is exposed to the alternating magnetic field which activates K_{ATP} channels thereby effecting vascular relaxation as further detailed hereinabove. In this embodiment, suitable filed intensities at skin level are, without limitation, from about 40 nT to about 300 nT, but other intensities are not excluded from the scope of the present invention.

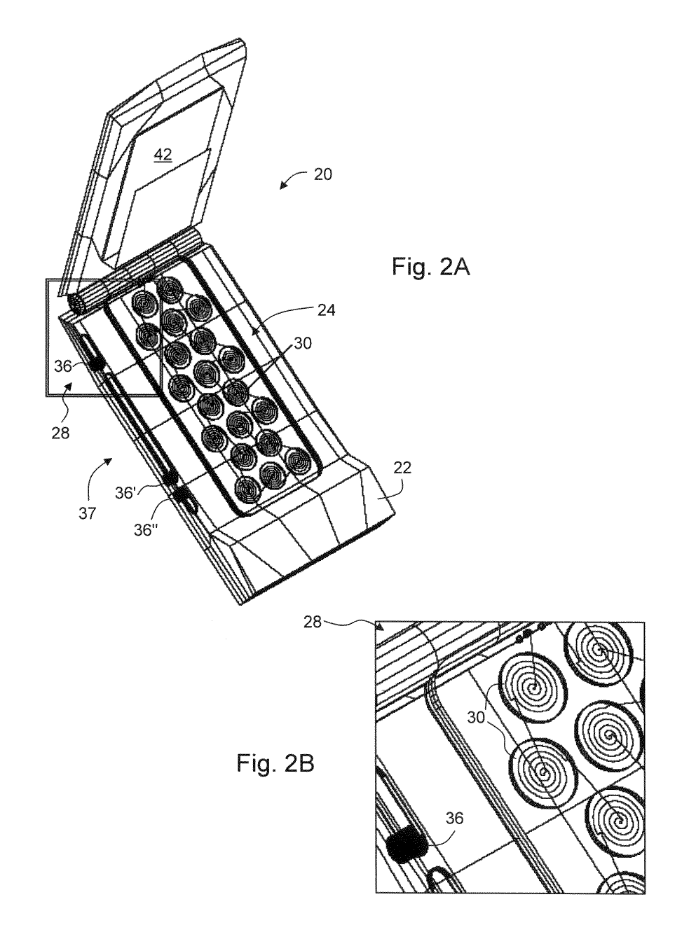

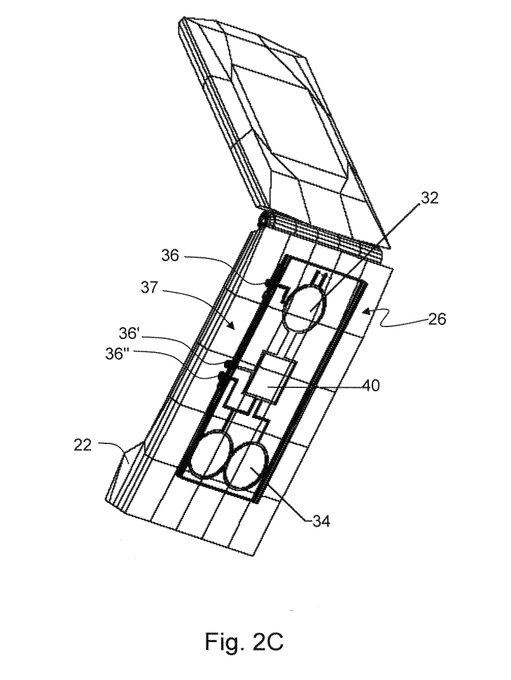

Device 10 can include an integrated circuitry and a power source (not shown, see, e.g., FIGS. 2A-C) or it can be provided with a separate power source and circuitry unit 13 which can be hand held, as shown in FIG. 1C, or mounted, e.g., on a limb of the subject, as shown in FIG. 1D. Power source and circuitry unit 13 serves for supplying alternating current to the coil to thereby generate the alternating magnetic field as further detailed hereinunder.

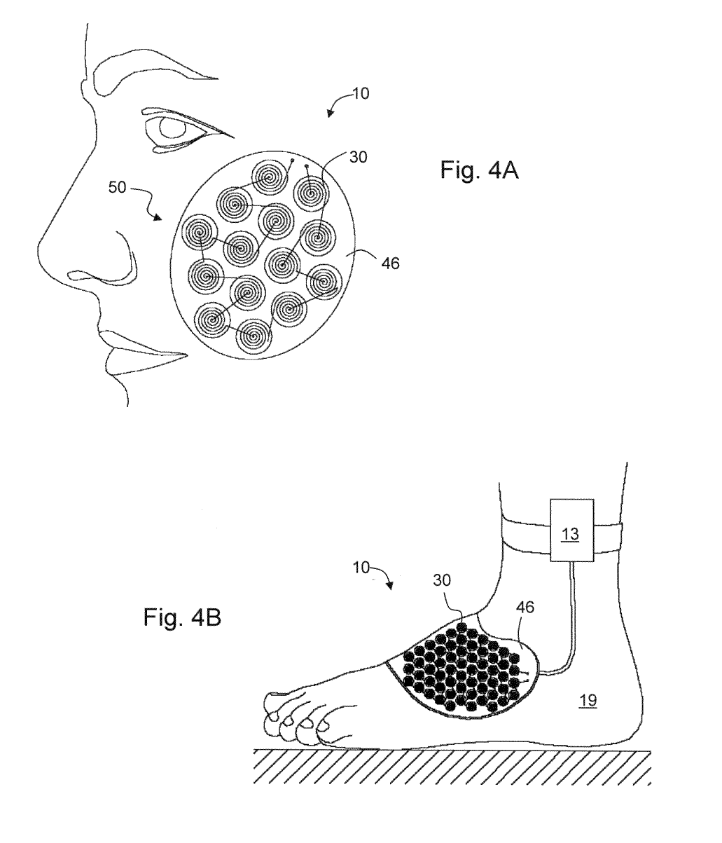

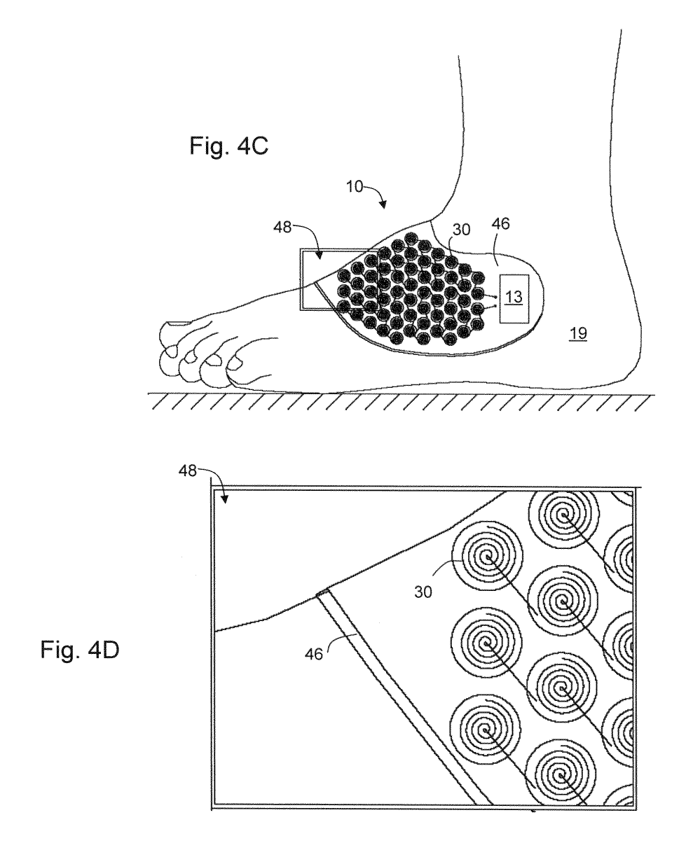

Device 10 can have any shape. In the representative example of FIGS. 1A-C device 10 is shaped as a ring on which a single coil is wound, but this need not necessarily be the case since it may be desired to have other shapes for device 10. For example, device 10 can be provided in the form of an attachable patch as further detailed hereinbelow (see FIGS. 4A-D).

Reference is now made to FIGS. 2A-C, which are schematic illustrations of a mobile system 20 according to various exemplary embodiments of the present invention.

System 20 comprises a cellular telephone unit 22 and an alternating magnetic field generator 24 configured for generating an alternating magnetic field at a frequency of from about 15.5N Hz to about 16.5N Hz, where N is a positive integer, as further detailed hereinabove. System 20 can be utilized to implement a simple and safe process of enhancing skin blood perfusion, and as a result endow the skin with an additional tint and a more appealing complexion. Such effect is realized through facial exposure to the alternating magnetic field of the present embodiments so as to activate potassium ATP channels thereby to effect vascular relaxation. As further detailed hereinabove, such vascular relaxation enhances blood perfusion, resulting in an increase in skin viability, and adding a moderate and desired blush.

The magnitude of the generated alternating magnetic field can be any of the aforementioned values. Preferably, but not obligatorily, when system 20 is held near the face, the skin is exposed to an alternating magnetic field having RMS intensity of from about 40 nT to about 300 nT.

The alternating magnetic field of the present embodiments preferably penetrates into the skin for a depth of few mm. It was found by the present Inventors that such superficial penetration is sufficient for inducing vasodilation in dermal arterioles via activation of potassium ATP channels as further detailed hereinabove.

The field is preferably sufficiently weak (as stated preferably about 40-60 nT at skin level) such that it significantly decays before reaching the brain tissue. It was further found by the Present Inventors that even when the alternating magnetic field of the present embodiments is applied to one side of the face (e.g., by holding system 20 close to the right cheek), the contra-lateral cheek can also gain similar effect through the mechanism of “entrainment” promoted by bilateral neural reflex arcs.

Besides inducing dermal microvascular dilation, the use of system 20 can also render the cells of the facial skin more resistance against the deleterious effects caused by solar radiation and/or smoking. Additionally, the use of system 20 can result in local elevation of skin temperature at the location where the alternating magnetic field is applied. The temperature is typically increased by from about 1.1° C. to about 1.7° C. Following a small delay (typically a few minutes or less) the effects are also observed in the contra-lateral cheek. In normal climate conditions, the dual effects can last more than an hour, e.g., two hours or more following termination of the alternating magnetic field.

Cellular telephone unit 22 can be any cellular telephone unit known in the art. The operation of unit 22 is preferably, but not obligatorily, independent of the operation of generator 24. Specifically, the user may use system 20 as a cellular telephone without activating the alternating magnetic fields, or he/she can use system 20 for generating alternating magnetic field without operating the cellular telephone. Also contemplated is a combined operation mode in which case the alternating magnetic field is generated while the user operates the cellular telephone.

Generator 24 is preferably integrated into the encapsulation of unit 22 and may comprise a miniature electronic circuitry 26 and an array 30 of coils, designed and constituted to generate the alternating magnetic field of the present embodiments. The coils of array 30 are better illustrated in FIG. 2B which is an enlarged view of a section designated by reference numeral 28 in FIG. 1A. A typical number of coils in array 30 is from about 10 coils to about 80 coils, more preferably from about 20 coils to about 60 coils. The typical diameter of each coil is from about 4 mm to about 20 mm. The coils can be connected serially via an arrangement of connecting wires, such that the same electrical current flows in each coil. For example, the central winding of each coil can be connected to the outermost winding of the adjacent coil thus forming a serial connection between all coils. Connection between coils is preferably established via printed circuit board technology as known in the art.

The typical number of windings in each coil is from about 5 windings to about 10 windings.

When the coils are identical in terms of dimension, material and number of windings, each coil generates substantially the same alternating magnetic field, and an overall alternating magnetic field generated by generator 24 is substantially synchronously uniform over the area of the generator. Generator 24, including array 30 and circuitry 26 can also be made detachable from unit 22 to allow generator 24 to function independently and as a separate unit.

FIG. 2C illustrates a back side of system 20, showing circuitry 26. In various exemplary embodiments of the invention can comprise circuitry 26 comprises an oscillator 32 for generating alternating current in coils 30 at magnitude and frequency suitable for generating the alternating magnetic field as described above and a power source 34 for powering oscillator 32. Power source 34 can comprise, for example, paper thin batteries (e.g., 2-3V batteries) or it can comprise the power source of unit 22 (not shown).

In various exemplary embodiments of the invention circuitry 26 comprises a user interface 37 for allowing the user to control (select and/or adjust) one or more of the alternating magnetic field parameters (intensity, frequency, alternating wave shape). In the representative illustration of FIGS. 2A-C user interface 37 is manufactured as a plurality of knobs 36, 36′ and 36″, positioned on the side of cellular unit 22. Knob 36 can facilitate control of the frequency, e.g., by adjusting the operation of oscillator 32, knob 36′ can facilitate control of the alternating magnetic field intensity, e.g., via an adjustable resistor 40 as known in the art, and knob 36″ can be an on/off knob which can be operated by the user to activate and deactivate generator 24, e.g., by establishing or disestablishing electrical connection between source 34 and oscillator 32. Other positions and types of user interfaces and/or additional user interface units are also contemplated. For example, user interface 36 can comprise a display (not shown) and/or a remote control unit (not shown) as known in the art. When generator 24 is detachable from unit 22, user interface 36 or part thereof can also be detachable. Circuitry 26 can also communicate with a display 42 of cellular unit 22 for providing the user with information regarding the generated field, exposure time and the like.

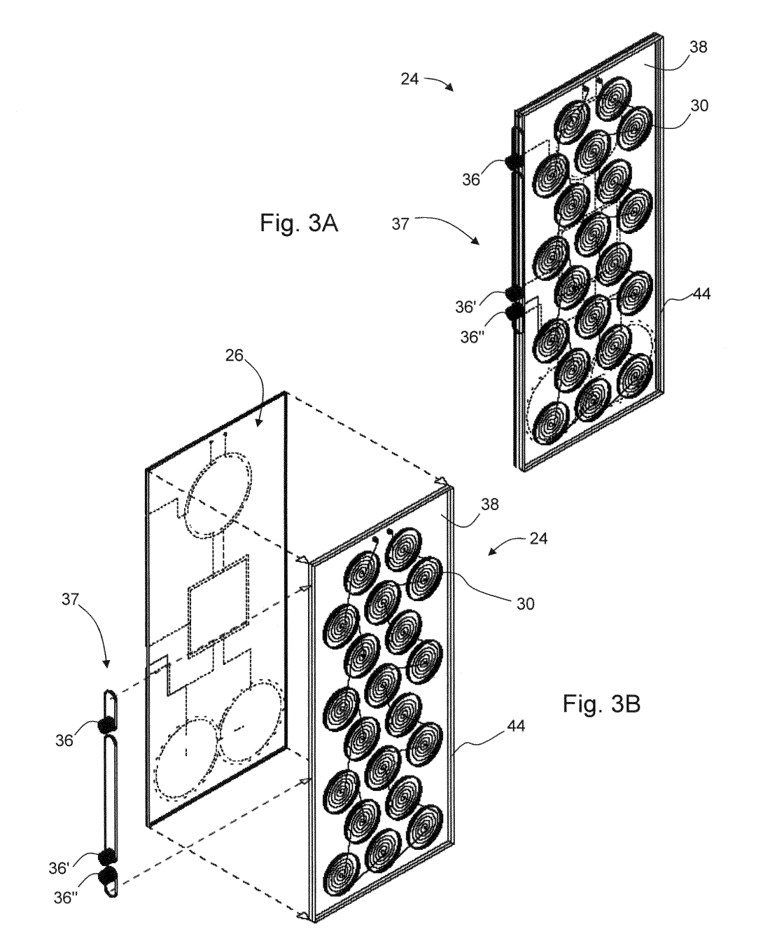

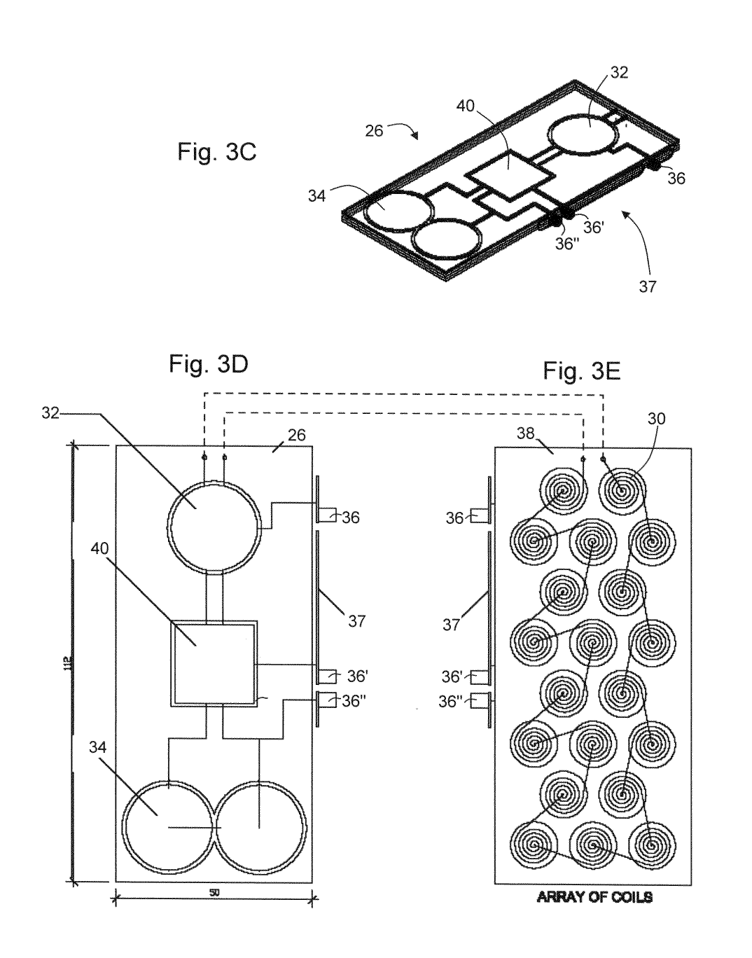

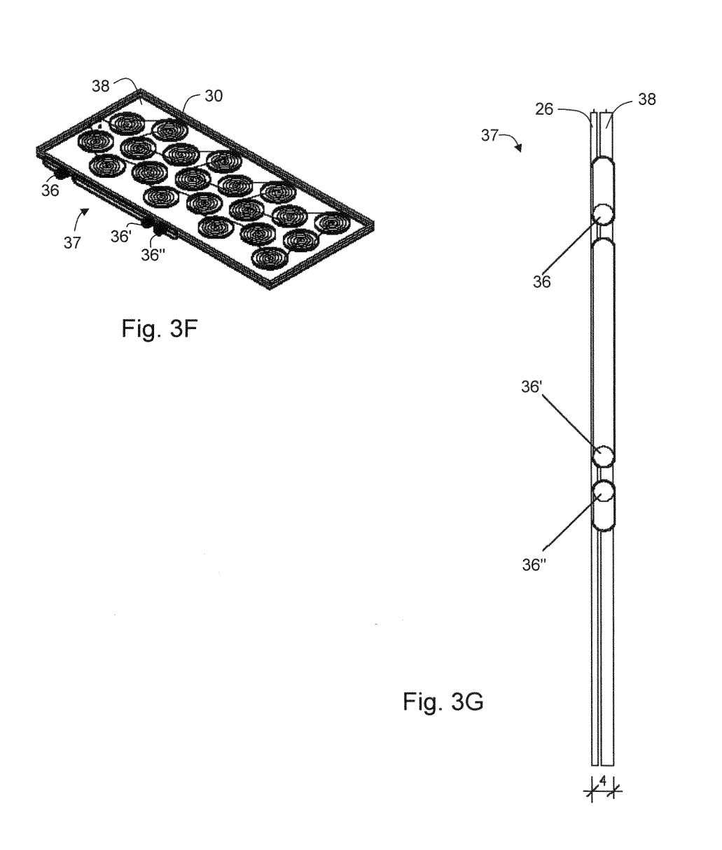

Generator 24 is preferably shaped as or being integrated in a substantially flat surface. This is particularly useful when generator 24 is detachable from unit. This embodiment is better illustrated in FIGS. 3A-G, showing a substrate 38 carrying coils 30 on its front side (FIGS. 3A, 3E and 3F), circuitry 26 on its back side (FIGS. 3B, 3C and 3D), and user interface 37 on its side (FIG. 3G). Generator 24 can be coated by a thin non-ferromagnetic partition 44. When generator 24 operates as a separate unit, partition 44 can be placed on the skin and the alternating magnetic field generated by the coils can penetrate 44 and treat the skin. Partition 44 is preferably, but not obligatorily, soft and pliable partition so as to facilitate easy placement on the skin.

The typical thickness of generator 24 is from about 1 mm to about 10 mm, more preferably from about 1 mm to about 5 mm. The planar dimensions of generator 24 are preferably sizewise compatible with the encapsulation of cellular unit 22. In use, generator 24 can be held (with or without unit 22) in a close proximity with the skin of the cheek and generally in parallel thereto so as to expose the cheek to the generated alternating magnetic field. Typically, but not obligatorily, the exposure to alternating magnetic field is for at least 10 minutes, more preferably at least 15 minutes or more.

In various exemplary embodiments of the invention the coils of array 30 are made of a conductive material such as copper, silver or any other conductive alloy suitable for generating an alternating magnetic field when electrical current flows therein.

Reference is now made to FIGS. 4A-D which are schematic illustration of medical device 10 in embodiments in which device 10 comprises an attachable patch. In this embodiment, device 10 preferably comprises a patch 46 which can be attached to the skin of the subject, array 30 of coils and a power source and circuitry unit 13 (not shown in FIG. 4A, see FIGS. 4B and 4C). The principles and operations of array 30 and unit 13 of device 10 are similar to the principle and operations of the coils, power source and circuitry of system 20 described hereinabove. The connections between the coils in array 30 is better illustrated in FIG. 4D which is an enlarged view of section 48 of FIG. 4C. Unit 13 can be integrated into patch 46, as shown in FIG. 4C, or it can be provided as a separate unit as shown in FIG. 4B. Unit 13 may or may not include a user interface, as desired. Typically, but not obligatorily patch 46 is disposable. The shape and size of patch 46 is preferably selected in accordance with the shape and size of the organ to which it is to be attached. In the representative illustration of FIG. 4A, patch 46 has a generally round shape which is suitable, for example, for attachment on a cheek 50 of a subject. In the representative illustrations of FIGS. 4B-D, patch 46 has a generally oval shape which is suitable, for example, for attachment on a foot 19 of a subject.

In various exemplary embodiments of the invention the back side of patch 46 is made of or being coated with an adhesive to allow attachment of patch 46 to the skin of the subject. The adhesive can be, for example, of the type used for attaching wound dressing or the like. Preferably, the amount and type of adhesive is selected so as to allow adherence between the patch and the skin for at least 10 minutes, more preferable at least 20 minutes, more preferably an hour or more.

When unit 13 comprises a user interface, it can include any of the elements described above. When unit 13 does not comprises a user interface, it can be automatically activated upon attachment of patch 46 the skin. For example, patch 46 can be packed such that when patch 46 is enclosed in the package there is no electrical connection between the power source and the coils, but when the package is removed or the adhesive cover is peeled, the electrical connection is established and the coils are being fed with current. Such packaging techniques are well known to those ordinarily skilled in the art. Once treatment is completed, patch 46 can be detached from the skin so as to terminate the exposure of the skin to the alternating magnetic field. Once removed, the user can discard the patch and/or manually disconnect the power source from the coils.

In various exemplary embodiments of the invention patch 46 comprises one or more medicament incorporated therein for intradermal or transdermal delivery of the medicament to the subject. Incorporation of the medicaments can be by any technique known in the art, including, without limitation, immersing the patch in a solution containing the medicament, manufacturing the patch from drug loaded fibers and the like. Preferably, the adhesive side of the patch is incorporated with the medicament, so as to facilitate intradermal or transdermal delivery of the medicament upon attachment of the patch to the skin.

For example, the patch can be incorporated with a potassium channel opener drug. In this embodiment, when the patch is attached to the skin and the coils generate the alternating magnetic field of the present embodiments, both the alternating magnetic field and the potassium channel opener drug activate the potassium ATP channels resulting in a combined effect. It was found by the Inventors of the present invention that the combination of administration potassium channel opener combine to a synergistic effect, because the alternating magnetic field, beside acting as potassium channel opener, also enhances the delivery of the drug to the skin vasculature.

One type of potassium channel opener is minoxidil. When used in a solution, emulsion, cream or any other formulation with concentration of 5% or more, minoxidil is known to have favorable effects on the growing hairs. Thus, the patch of the present embodiments can be incorporated with minoxidil, preferably at concentration of 5% or more. This embodiment is particularly useful for treating alopecia or alopecia areata. In use, the patch can be attached to the scalp and the coils can be activated. The minoxidil is gradually released from the patch and, synergistically with the effect of the alternating magnetic field, can encourage hair growth on the scalp.

The patch of the present embodiments can also be incorporated with other drugs, such as, but not limited to, antibiotic or antiseptic medications. This is particularly useful when the patch is used for treating skin cuts or wounds. In use, the antibiotic or antiseptic medications are gradually released to the skin. Both the released medications and the alternating magnetic field of the present embodiments act in combination to treat the wounds. The alternating magnetic field enhances dermal blood perfusion, skin healing and facilitate better delivery of the medicament to the wound site. Such synergistic effect, of increase in local blood perfusion together with medical agents, can rapidly and favorably heal a cut-wound as to eventually leave a minimal residual scar, a situation which is of utmost importance particularly in the event of facial skin.

The present embodiments successfully provide a shielding device for the protection of individuals against alternating magnetic fields of low frequency (e.g., up to 300 Hz, more preferably from about 40 Hz to about 70 Hz) which prevail in common households and in the vicinity of power stations and power lines. The shielding device of the present embodiments is particularly useful for individuals which are at risk of developing complications due to exposure to such alternating magnetic field. Specifically, but not exclusively, the shielding device of the present embodiments is useful for individuals possessing heart failure, pregnant women, embryos and infants.

The present Inventors found that electricity networks, which typically operate at frequencies of about 50-60 Hz, may activate myocardium K_{ATP} channels at the cell membrane and promote calcium efflux from the myocytes. This is because these frequencies are close to integer multiplications (higher harmonics) of the open-close frequencies associated with K_{ATP} channels. For example, an individual being in close proximity to an electrical network or electrical appliance operating at a frequency of 50 Hz, may experience elevated potassium activity because the electrical network or electrical appliance generates an electromagnetic wave having a magnetic component of 50 Hz which is close to the third harmonic (48 Hz) of 16 Hz. Similarly, an individual being in close proximity to an electrical network or electrical appliance operating at a frequency of 60 Hz, may experience elevated potassium activity because a magnetic component of 60 Hz is close to the fourth harmonic (64 Hz) of 16 Hz.

As described above, activation of potassium ATP channels effects an efflux of calcium ions from the myocytes. For individuals exhibiting symptoms of heart failure and embryos, the effect of calcium ion depletion from the already compromised cells can be detrimental. Individuals in congestive heart failure and embryos are very sensitive to calcium ion depletion since this process can precipitate a bout of severe failure which may result in stressful medical condition.

The activation of K_{ATP} channels in the myocardial cell membrane, in patients suffering from CHF, induces an adverse effect by the reduction in the minimal concentration of intracellular calcium in the compromised myocytes. The shield device of the present embodiments can be used to substantially preserve the contraction ability of the heart.

It was found by the present Inventors that there are changes in ECG at power station facilities and at sufficiently small distance from common electric appliances or public transformers (up to abut 20-30 centimeters from low-power appliances and up to about 50 centimeters for high power transformers). The ECG changes observed by the present Inventors were typical for activation of K_{ATP} channels. These changes were manifested as amputations or reduction in the amplitude of the R wave in the ECG QRS complex. In a search for protective means against such effect, the present Inventors devised a shielding device which substantially prevents electromagnetic radiation from activating K_{ATP} channels.

Patients who are under therapy or follow-up for congestive failure are expected to benefit from the shielding device of the present embodiments because the device can effect blocking of excessive calcium ions efflux from the myocardial cells. Patients in congestive heart failure are very sensitive to calcium ion depletion. Intracellular calcium governs the contraction of myocardial cells and undue calcium loss may induce a deleterious effect on myocardial contraction in patients with heart failure resulting in a possible event, critical cardiac dysfunction and curtailed cardiac output.

The activation of K_{ATP} channels by magnetic fields of certain frequencies (close to integer multiplication of 16 Hz) mimics the effect of calcium channel antagonists such as verapamil, diltiazem or other calcium channel blockers. While the calcium channel blockers have direct effect on L-type Ca2+ channels, a magnetic field of certain frequency may promote such effect indirectly, by activation of K_{ATP} channels as described above.

Verapamil, for example, is a medication which is typically prescribed for patients suffering from hypertension. Yet, the use of verapamil during pregnancy has been limited by child-cardiologist due to the experimentally proven detrimental effect of the calcium channel block on the embryo's heart. Thus, when alternating magnetic field having a frequency which is sufficiently close to an integer multiplication of 16 Hz mimics the effect of verapamil on pregnant woman, it can be hazardous to the heart of embryo.

The shielding device of the present embodiments can therefore be used by pregnant women so as to substantially prevent undesired activation of K_{ATP} channels in the myocytes of the embryo's myocardium which results in depletion of calcium ions from the myocytes.

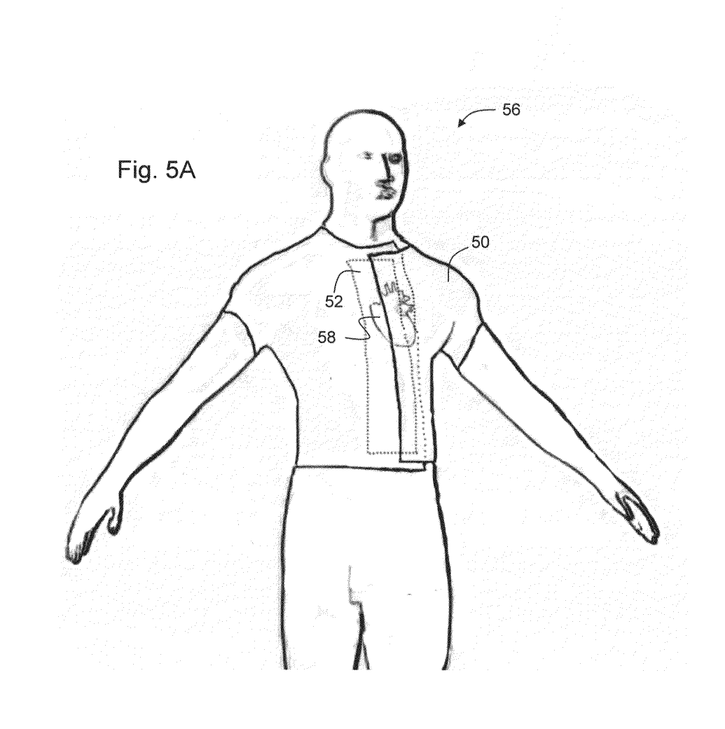

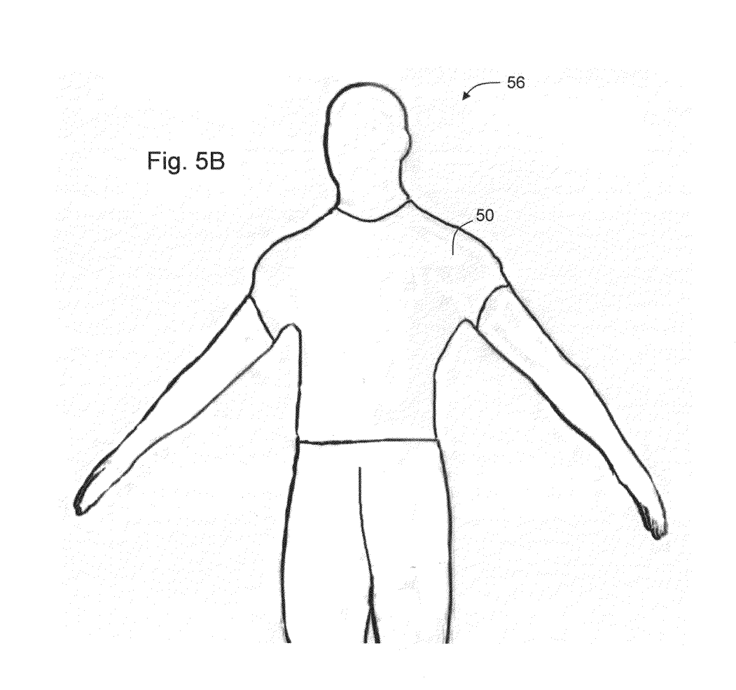

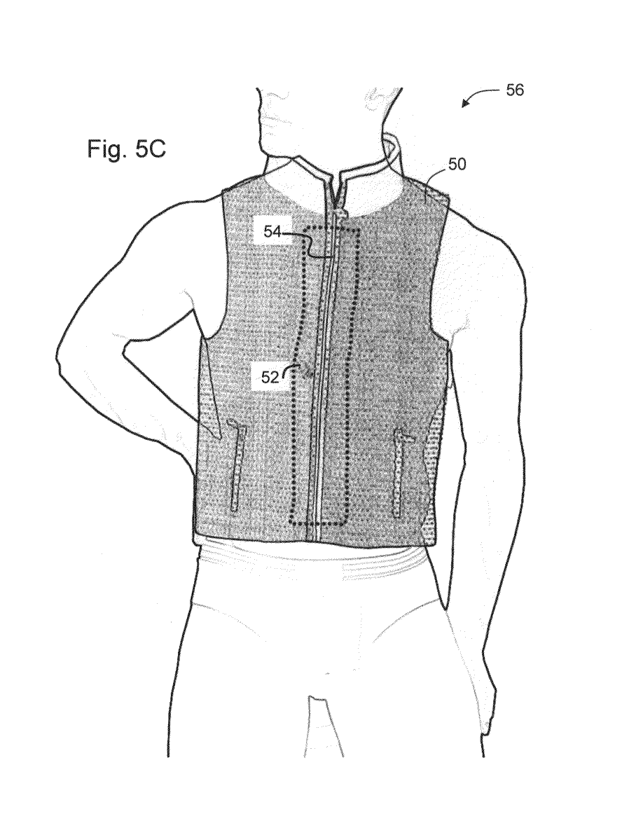

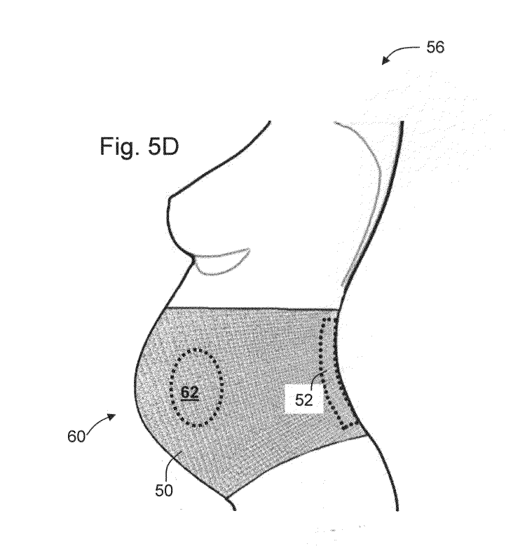

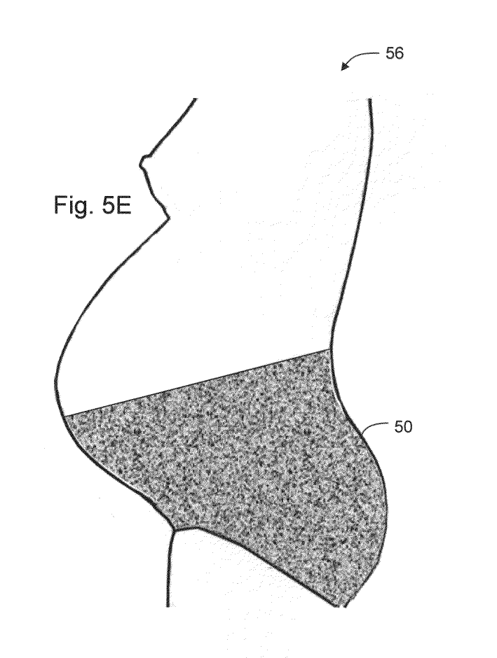

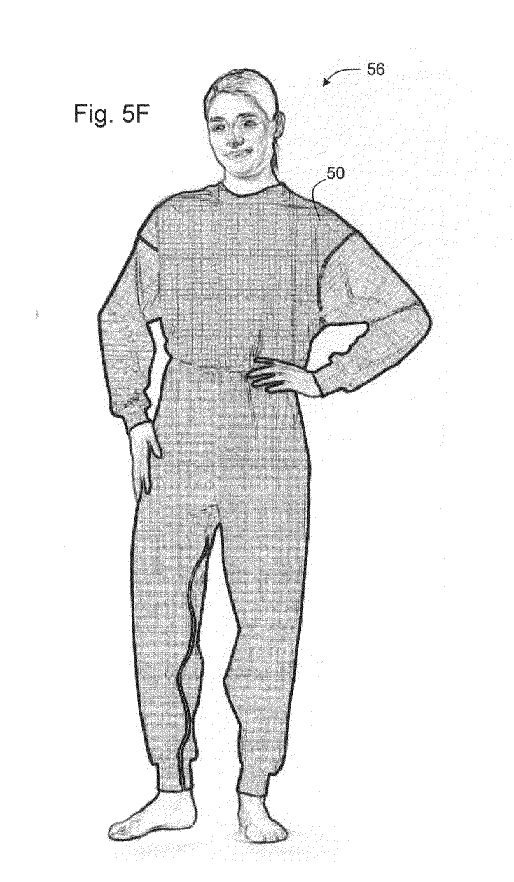

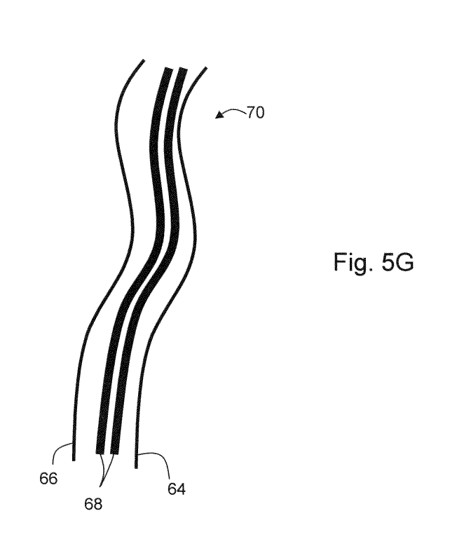

Reference is now made conjointly to FIGS. 5A-F, which schematically illustrate a shielding device 50 according to various exemplary embodiments of the present invention. Device 50 comprises a ferromagnetic core having a closed shape and being embedded in a garment designed to be worn by a subject 56 and cover an organ thereof. The ferromagnetic core of device 50 is designed and constructed to attenuate or substantially prevent penetration of an alternating magnetic field at low frequency (e.g., a frequency of less than 300 Hz, more preferably from 40 Hz to 70 Hz) therethrough. In various exemplary embodiments of the invention the ferromagnetic core attenuates and substantially prevents penetration of an alternating magnetic field at a frequency of from about 50 Hz to about 60 Hz which are typical frequencies in common electrical networks and home appliances.

FIG. 5G is a schematic illustration showing fragmentary view of a cross section of device 50. Shown in FIG. 5G is a garment 70 made of two layers 66 and 64, and a ferromagnetic core 68 interposed between layers 66 and 64 of garment 70. Garment 70 can be made of any fabric suitable for wearing. Also contemplated are fabrics incorporated with ferromagnetic beads or flakes.

The ferromagnetic core is preferably pliable and thin. For example, the ferromagnetic core can be made of one or more thin layers of ferrite or other metal such as iron alloys. The ferromagnetic core of device 50 can be made of a composition of ferric oxide with other material, such as, but not limited to, silicon, aluminum, magnesium, iron and the like. The ferromagnetic core is preferably made of substance characterized by the general formula M(FeₓO_{y}), e.g., M(Fe₃O₄), where “M” stands for any divalent metal, including, without limitation, nickel, magnesium, copper, cobalt and any combination thereof. Preferably, but not obligatorily the core includes less than 50% iron.

Typical thickness of the core is, without limitation from about 0.0006 inches to about 0.004 inches.

The ferromagnetic core can have a ceramic-like construction having ferromagnetic properties. For example, a ceramic-like construction can be obtained by the addition of certain amount of iron nickel or other ferromagnetic substance.

The ferromagnetic core can also be constructed of polycrystalline. For example, the core can be made of a plurality of iron oxide crystalline structures or similar materials, including, without limitation, magnesium, aluminum, barium, manganese copper, nickel, cobalt and any combination thereof. The crystalline structures may posses any configuration, including, without limitation, spinet, garment and perovskite hexagonal.

When the ferromagnetic core comprises two or more layers, each can be made of a different ferromagnetic material, thickness and/or structure. In this embodiment, each layer can be optimized to shield a different field level in a predetermined arrangement. For example, the layers can be arranged such that the first (outer) layer is optimized for the highest field level, the second layer is optimized for a lower field level and so on. Spacing can be formed between adjacent layers to increase attenuation.

The ferromagnetic core or each layer thereof can be made of a continuous surface, or it can be made of a plurality of adjacent and joined stripes. Also contemplated is a ferromagnetic core structured as a mesh of ferromagnetic wires. In this embodiment, the mesh spacing is selected sufficiently small to prevent penetration of the electromagnetic field therethrough.

Device 50 is utilized to implement a simple and safe protection against electromagnetic radiation, which is typically weak. Typically, the intensity of the magnetic component of the radiation is in the sub microtesla range.

In various exemplary embodiments of the invention shape and size of the garment is selected to protect the heart 58 of the subject from the alternating magnetic field (FIGS. 5A-C). In other embodiments the shape and size of the garment is selected to protect the abdomen 60 of a pregnant woman, hence the heart of the embryo 62 carried thereby (FIG. 5D-E). Also contemplated are garment protecting other organs or the entire body (FIG. 5F).

Representative examples of garments suitable for shielding device 50 include, without limitation, a shirt, a coat, a vest, underwear, a suit, an overall and the like. Device 50 can have a permanently closed shape (such as in the case of a T shirt, for example) or it can be provided as an open garment in which case the device preferably includes one or more connecting fasteners 54 (see, e.g., FIG. 5c) to allow the user to fasten the garment following wearing. In various exemplary embodiments of the invention device 50 comprises one or more ferromagnetic strips 52 for ensuring continuity of the shield. Strip 52 can be used, for example, when the garment has connecting fasteners 54, in which case strip 52 is preferably placed such that it fully overlaps the fasteners.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

The inventors observed counteracting effect of the agent glibenclamide (a specific blocker of channel activation) on calcium ions efflux from myocytes in cultures following exposure exposed to low intensities of 16 Hz magnetic field on K_{ATP} channels. This substantiates that closure of the L-type calcium channels at the level of the cellular membrane is a secondary process to the activation of the channels, resulting in significant reductions in intra-cellular calcium concentrations, a process that can be blocked or greatly diminished throughout the period that the blocking agent has its ongoing effect.

In vitro and in vivo experiments, in which the myocytes were exposed to alternating magnetic fields having frequencies other than 16 Hz or integer multiplication thereof have not yielded particular response. Some reduced effects were observed with frequencies of 50 Hz and 60 Hz, which are close to the third and fourth harmonics of 16 Hz.

Following is a description of experiments performed by the present Inventors to investigate the physiological of the alternating magnetic field of the present embodiments.

### Example 1

### The Effect of 16 Hz Magnetic Fields on Rat's Heart

### In Vivo Exposure to Alternating Magnetic Field

Male Sprague Dawley rats (N=36) weighing 237±14 g were anesthetized using intramuscular injection of Xylazine (10 mg/kg) and Ketamine (90 mg/kg). The limbs of the tested animals were shaved and cleaned with alcohol. Electrodes were gently fixed around the shaved limbs. ECG (from limb electrodes) was continuously recorded for 60 minutes using Biopac (England). Following 15 minutes of base line, the rats were exposed to 20 minutes field of 80 nT and different frequencies, until stable measurement were achieved. In order to avoid extra injection of anesthesia, the ECG was no later than 25 minutes during recover time. P, QRS, and T duration (ms) and amplitude (mV), and QT duration (ms) were measured automatically during the experiments using the Biopac software.

### Statistical Analysis of ECG Data

Baseline data from ECG parameters, before application of alternating magnetic field were normalized to 100 percent and all values are presented as percent change from baseline. Mean and SD were calculated. A P≦0.05 was considered statistically significant

### Preparation of Isolated Cardiac Myocytes

Cardiac myocytes were extracted in order to study intracellular free calcium ([Ca²⁺]ᵢ) shifts as a result of exposure to alternating magnetic field. This was estimated from indo-1 fluorescence using the ratio method.

Rat hearts (2-3 days old) were removed under sterile conditions washed three times in phosphate buffered saline (PBS) to remove excess blood cells. The hearts were minced and then gently agitated in a solution of proteolytic enzymes, RDB. The RDB was diluted 1:100 in Ca²⁺ and Mg²⁺ free PBS at 25° C. for a few cycles of 10 minutes each, Dulbecco's modified Eagle's medium (DMEM) supplemented with inactivated 10% horse serum (Biological Industries, Kibbutz Beit Haemek, Israel), and 0.5% chick embryo extract was added to the supernatant suspensions containing dissociated cells. The mixture was centrifuged at 300 g for 5 minutes. The supernatant phase was discarded and the cells were resuspended in the same medium. The suspensions of the cells were diluted to 1.0×10⁶ cells/ml and were placed in 35-mm plastic culture dishes on collagen/gelatin coated cover glasses. The culture was incubated in a humidified atmosphere of 5% CO2, 95% air at 37° C. All experiments were performed at day 4 in culture.

### Exposure of the Isolated Cardiac Myocytes to Alternating Magnetic Field

In a first set of experiment cardiac myocytes were exposed to 10 minutes of base line, 20 min of alternating magnetic field (80 nT and 16 Hz) in order to achieve stabilization of the amplitude followed by 40 minutes of recovery.

In a second set of experiments the specificity of the myocyte ion channels (K_{ATP}) was tested by inducing exposure alternating magnetic field of 16 Hz, that were delivered for 10 minutes of base line (n=10). Following baseline recording, the channels were blocked by the addition to the dish 5 μM of Glibenclamide and alternating magnetic field was delivered for an additional 20 minutes.

In the third setup of the experiment the amount of 5 μM of Glibenclamide was primarily added (n=10), to be followed by exposure to alternating magnetic field of 20 min.

In the fourth phase of the experimental setup 3 μM of Nicorandil, a potassium channel opener (KCO) were added (n=10), following 10 minutes, alternating magnetic field was exposure was introduced for 20 minutes.

In the fifth phase of the experiment alternating magnetic field was applied for 20 minutes followed by introduction of 3 μM of Nicorandil (n=5).

In addition, the effect of alternating magnetic field on Ca² transient was investigated using frequencies other than 16 Hz (8 Hz, 15 Hz, 17 Hz, 32 Hz, 50 Hz, 60 Hz).

In all experiments, the alternating magnetic field intensity was measured using a miligauss magnetometer (Gaussmeter “Alphalab”, Salt Lake City, USA).

### Intracellular Calcium Measurements

Intracellular free calcium concentration [Ca²⁺], was estimated from indo-1 fluorescence using the ratio method previously described before by Grynkiewicz et al (1985). Following incubation, the cells were rinsed twice with glucose enriched PBS and transferred to a chamber on the stage of a Zeiss inverted epifluorescent microscope. Indo-1-AM was excited at 355 nm and the emitted light was then split by a dichroic mirror to two photomultipliers, with input filters at 405 and 495 nm. The fluorescence ratio of 405/495 nm, which is proportional to [Ca2+] was fed into a computer program. The time integral of Ca²⁺ Influx was determined as area under the curve via a program, which gives the integral during any specified time window. From this value the baseline offset, measured at the systolic and diastolic phase, were subtracted.

### Statistical Analysis of Calcium Data

Baseline data from calcium transients were normalized to 100 percent and all values were presented as percent change from baseline. Mean and SD were calculated. A P≦0.05 was considered statistically significant.

### Results

All rats survived the in vivo experiments. The ECG parameters of 36 rats were collected and grouped together. Significant changes were achieved with WMF with a frequency of 16 Hz having intensity of 80 nT.

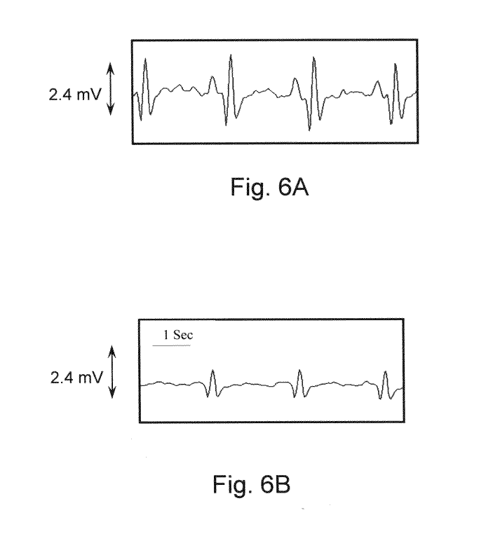

FIGS. 6A-B show examples of a typical ECG changes observed in a rat. Shown are base-line ECG (FIG. 6A) and following 15 minutes exposure to the alternating magnetic field of the present embodiments. Shortening QRS amplitude, P and T waves were observed.

Tables 1 and 2 below present a summary of ECG changes (% change from base line) in rats using various alternating magnetic field frequencies.

TABLE 1

| | P | QRS | QRS | T | T | QT |
|---|---|---|---|---|---|---|
| | Amplitude | Duration | Amplitude | Duration | amplitude | Duration |
| 60 Hz; | −4.3% ± 2.1% | −3.7% ± 2.3% | −6.4% ± 2.2% | +2.4% ± 2.2% | −11.7% ± 2.2% | −2.2% ± 3.1% |
| 80 nT | (p = ns) | (p = ns) | (p = 0.04) | (p = ns) | (p = ns) | (p = ns) |
| (n = 4) | | | | | | |
| 50 Hz; | +1.3% ± 4.1% | −4.7% ± 23% | −5.4% ± 2.2% | −5.4% ± 6.2% | −11.7% ± 7.2% | −3.8% ± 3.1% |
| 80 nT | (p = ns) | (p = ns) | (p = ns) | (p = ns) | (p = ns) | (p = ns) |
| (n = 4) | | | | | | |
| 15.95; | −13.3% ± 4.1% | −7.7% ± 3.3% | −15.4% ± 4.2% | −3.4% ± 6.2% | −23.7% ± 7.2% | −8.8% ± 3.1% |
| 80 nT | (p = 0.01) | (p = 0.03) | (p = 0.001) | (p = 0.01) | (p = 0.001) | (p = 0.02) |
| (n = 12) | | | | | | |
| 22 Hz; | 3.13% ± 4.1% | +2.7% ± 1.3% | −2.8% ± 2.2% | −1.2% ± 1.2% | +2.7% ± 4.2% | −2.8% ± 1.1% |
| 80 nT | (p = ns) | (p = ns) | (p = ns) | (p = ns) | (p = ns) | (p = ns) |
| (n = 4) | | | | | | |
| 2 Hz; | −1.3% ± 1.1% | +2.7% ± 2.4% | −2.4% ± 1.2% | −1.4% ± 1.2% | +2.7% ± 1.2% | −2.8% ± 1.1% |
| 80 nT | (p = ns) | (p = ns) | (p = ns) | (p = ns) | (p = ns) | (p = ns) |
| (n = 4) | | | | | | |

TABLE 2

| | P | QRS | QRS | T | T | QT |
|---|---|---|---|---|---|---|
| | Amplitude | Duration | Amplitude | Duration | amplitude | Duration |
| 15.95 Hz | −13.3% ± 4.1% | −7.7% ± 3.3% | −15.4% ± 4.2% | −13.4% ± 6.2% | −23.7% ± 7.2% | −8.8% ± 3.1% |
| 80 nT | (p = 0.01) | (p = 0.03) | (p = 0.001) | (p = 0.01) | (p = 0.001) | (p = 0.02) |
| (n = 12) | | | | | | |
| 15.95 Hz | −11.3% ± 3.7% | −6.7% ± 4.3% | −13.4% ± 3.2% | −4.4% ± 5.2% | −24.7% ± 5.2% | −3.8% ± 2.1% |
| 8 nT | (p = 0.02) | (p = 0.03) | (p = 0.002) | (p = 0.02) | (p = 0.003) | (p = ns) |
| (n = 4) | | | | | | |
| 15.95 Hz | −9.3% ± 2.1% | −2.7% ± 1.3% | −8.2% ± 3.2% | −11.4% ± 6.2% | −15.7% ± 7.2% | −5.8% ± 3.1% |
| 0.8 nT | (p = 0.02) | (p = ns) | (p = 0.03) | (p = 0.01) | (p = 0.001) | (p = 0.025) |
| (n = 4) | | | | | | |

Table 3 below presents a summary of the effect of the alternating magnetic field of the present embodiments on Ca²⁺ transients in cardiac myocytes in culture using different alternating magnetic field frequencies and intensity of 80 nT.

TABLE 3

| | | | % Change from base | |
|---|---|---|---|---|
| Frequency | | n | line | P |
| 8 | Hz | 5 | −24% ± 5% | 0.04 |
| 15 | Hz | 5 | −7% ± 3% | ns |
| 15.95 | | 15 | 56% ± 7% | 0.002 |
| 17 | Hz | 2 | −5% ± 3% | ns |
| 32 | Hz | 3 | −15% ± 6% | 0.05 |
| 50 | Hz | 4 | −6% ± 2% | ns |
| 60 | Hz | 5 | −4% ± 1% | ns |

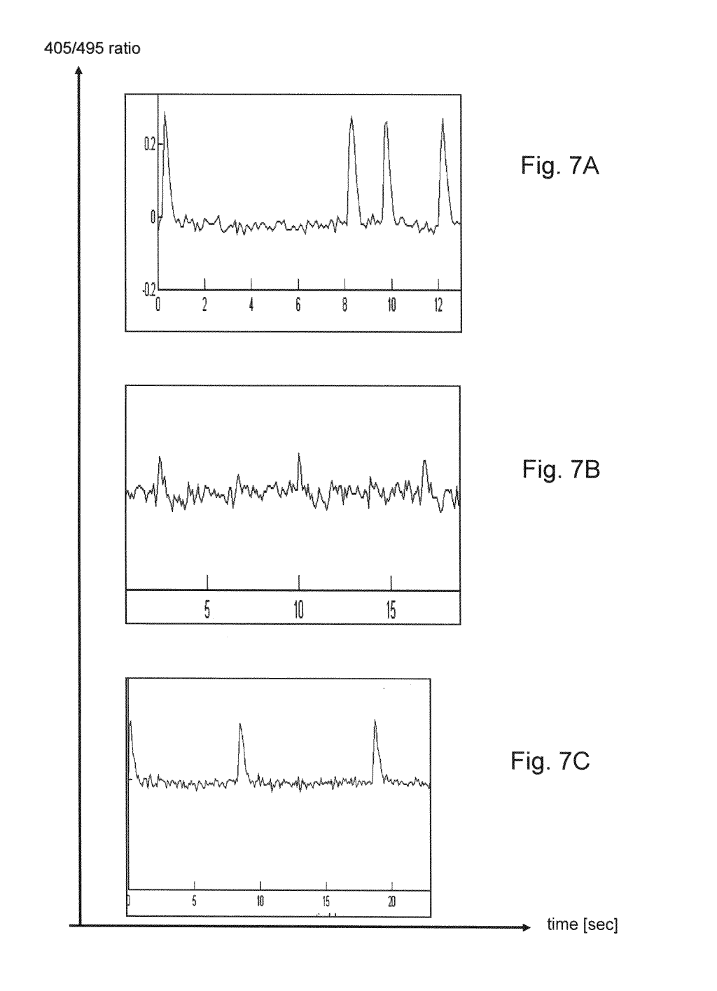

FIGS. 7A-C show changes in Ca²+ transients obtained from isolated cardiac myocytes during alternating magnetic field of 16 Hz. FIG. 7A shows control, FIG. 7B shows results during application of the alternating magnetic field and FIG. 7C shows recovery following termination of the applied alternating magnetic field.

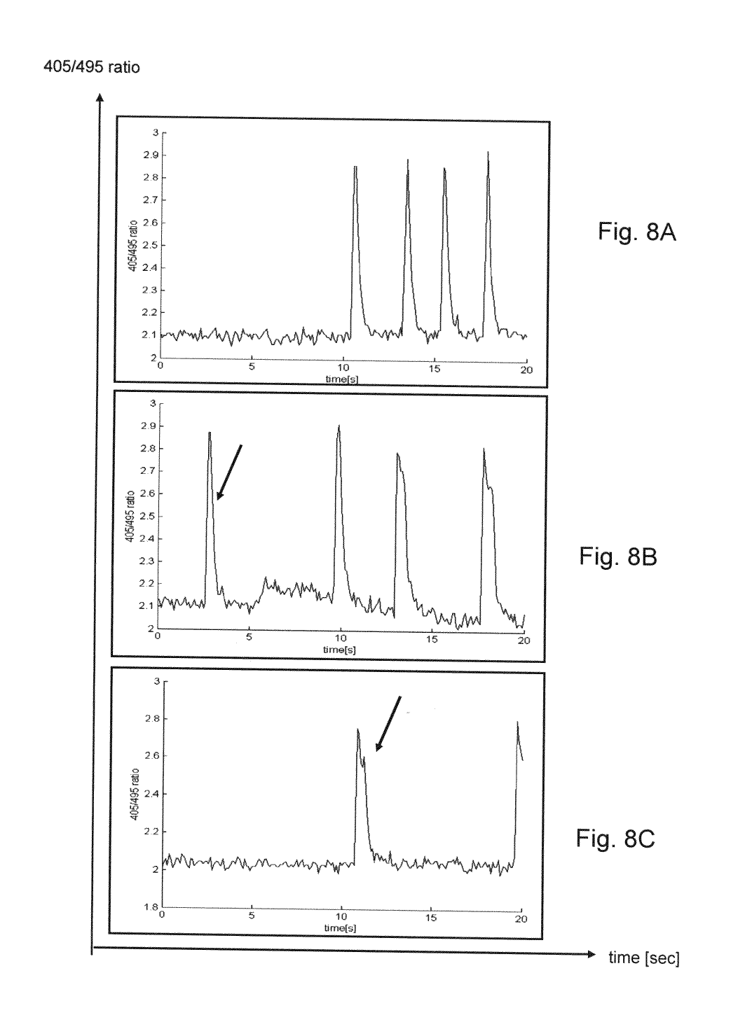

FIGS. 8A-C show changes in Ca²+ transients obtained from isolated cardiac myocytes with glibenclamide and during alternating magnetic field of 16 Hz. FIG. 8A shows control, FIG. 8B shows results with glibenclamide and FIG. 8C shows results following 20 minutes of application of the alternating magnetic field. The arrow in FIG. 8B points to a +30%±9% (n=10) widening of amplitude, and the arrow in FIG. 8C points to −24%±6% (n=10) narrowing of the amplitude.

FIGS. 9A-C show changes in Ca²+ transients obtained from isolated cardiac myocytes with Nicorandil and during alternating magnetic field of 16 Hz. FIG. 9A shows control, FIG. 9B shows results with nicorandil and FIG. 9C shows results following 20 minutes of application of the alternating magnetic field. The arrow in FIG. 9B points to a −58%±7% narrowing of amplitude, and the arrow in FIG. 9C points to −63%±3% (n=4) narrowing of the amplitude.

### Example 2

### The Effect of 16 Hz Magnetic Fields on Human Heart

In order to investigate the effect of alternating magnetic field of 16 Hz on ECG pattern on intact hearts of healthy human volunteers, male volunteers were subjected to the study (n=15). All ECG leads were connected to the examinees using 12 channel recorder (Philips, Germany). The alternating magnetic field of 16 Hz emitted from the activated coil were grossly perpendicular to the region of the heart under leads V₃-V₅. This resulted in apparent relatively greater exposure to the fields of the anterior, anteroapical and anterolateral regions of the myocardium. Female volunteers were not selected because of difficulties in placing the coil over the chest.

When activated, the coil emitted alternating magnetic fields of 3-5 μT. At a depth (distance) of 3.5-4.0 cm the fields were assumed to be 80-100 nanotesla at the gross location of the radiated location of the volume-region of the heart, as a result of physical laws the decline of field intensity was related to the increased distance from the coil emitting the alternating magnetic field.

The experimental protocol included 10-15 minutes of rest before activation of the field when baseline ECG records were obtained, followed by 10 minutes of field activation and 20 to 60 minutes to allow recovery.

The examinees were not aware at the moments of time when the field was activated or arrested. Continuous ECG records were obtained throughout the 3 stages of the experiment, and in 6 of the volunteers the ECG records were accompanied by echo Doppler and tissue-Doppler examinations. During the studies there were not any apparent changes in heart rate or blood pressure. The examinees did not feel any symptoms; no side effects were observed, and no arrhythmias even as elementary as premature beats were noted.

### Tissue Doppler Studies

Echo was performed using Philips 7500 ultrasound system on 6 healthy individuals. Measurements were performed for base line, 10 min. during alternating magnetic field and post alternating magnetic field application or at recovery. Standard 2-dimensional images, M mode, color Doppler, and Doppler tissue interrogation were performed in all 3 sessions. Peak early (E), late (A) transmitral velocities, E wave deceleration time, Doppler tissue myocardial velocities in systole (S) and early diastole (E′) and atrial contraction (A′) at the lateral and septal mitral annulus, were measured before, during, and after alternating magnetic field application.

### Statistical Analysis

Parameters were normalized to 100 percent and all values are presented as percent change from baseline. Values from Tissue Doppler measurements were compared between base-line and during alternating magnetic field application.

Mean and SD were calculated. A P≦0.05 was considered statistically significant.

### Results

Table 4 below presents a summary of ECG parameters in human using an alternating magnetic field having frequency of 15.95 Hz and intensity of 80 nT.

TABLE 4

| | | | | | During Application of | | | | | |
|---|---|---|---|---|---|---|---|---|---|---|
| | | Base Line | | | Magnetic Field [10 min] | | | Recovery [10 min] | | |
| | | QRS | | QT | QRS | | | QRS | T | QT |
| Pt # | Age | (mv) | T (mv) | (sec) | (mv) | T (mv) | QT (sec) | (mv) | (mv) | (sec) |
| 1 | 72 | 1.42 | 0.4 | 0.38 | 1.35 | 0.46 | 0.393 | 1.36 | 0.45 | 0.394 |
| 2 | 65 | 0.8 | 0.34 | 0.42 | 0.42 | 0.167 | 0.398 | 0.45 | 0.167 | 0.398 |
| 3 | 59 | 1.95 | 0.72 | 0.37 | 1.3455 | 0.504 | 0.382 | 1.35 | 0.5 | 0.381 |
| 4 | 73 | 0.72 | 0.18 | 0.42 | 0.648 | 0.15 | 0.41 | 0.648 | 0.15 | 0.41 |
| 5 | 70 | 1.6 | 0.53 | 0.38 | 1.376 | 0.556 | 0.365 | 1.377 | 0.557 | 0.365 |
| 6 | 62 | 1.75 | 0.39 | 0.358 | 1.54 | 0.395 | 0.362 | 1.53 | 0.396 | 0.362 |
| 7 | 65 | 2.45 | 0.35 | 0.362 | 2.32 | 0.3115 | 0.361 | 2.32 | 0.32 | 0.361 |
| 8 | 69 | 1.483 | 0.483 | 0.367 | 1.2 | 0.323 | 0.365 | 1.23 | 0.324 | 0.367 |
| 9 | 69 | 1.37 | 0.35 | 0.362 | 1.246 | 0.252 | 0.363 | 1.245 | 0.25 | 0.363 |
| 10 | 49 | 1.45 | 0.42 | 0.282 | 1.2325 | 0.407 | 0.281 | 1.233 | 0.41 | 0.281 |
| 11 | 69 | 1.53 | 0.41 | 0.362 | 1.33 | 0.393 | 0.362 | 1.3 | 0.394 | 0.363 |
| 12 | 31 | 0.9 | 0.37 | 0.366 | 0.765 | 0.385 | 0.366 | 0.764 | 0.395 | 0.367 |
| 13 | 28 | 2.3 | 0.38 | 0.412 | 1.748 | 0.4 | 0.396 | 1.75 | 0.42 | 0.398 |
| 14 | 37 | 2.1 | 0.42 | 0.387 | 1.66 | 0.441 | 0.376 | 1.67 | 0.4 | 0.378 |
| 15 | 31 | 1.86 | 0.22 | 0.42 | 1.6 | 0.227 | 0.43 | 1.6 | 0.442 | 0.42 |
| Av | 56.6 | 1.57 | 0.39 | 0.37 | 61.32 | 0.36 | 0.37 | 1.32 | 0.23 | 0.38 |
| SD | 55.5 | 1.58 | 0.12 | 0.035 | 0.46 | 0.11 | 0.03 | 0.46 | 0.11 | 0.03 |

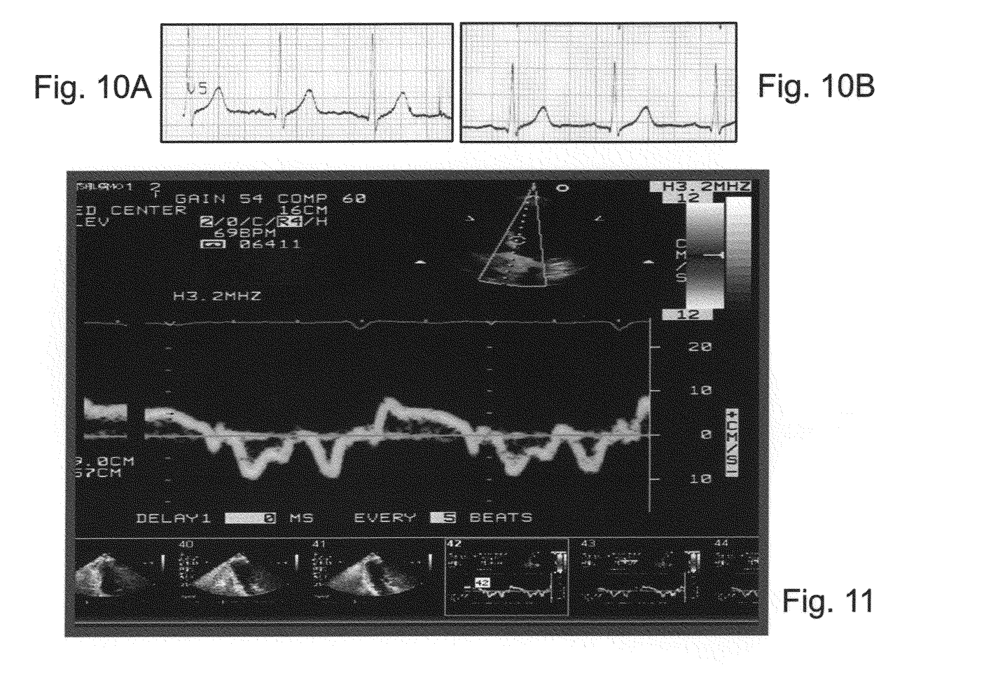

FIGS. 10A-B show examples of typical ECG changes observed in healthy human volunteer. Control record of the ECG is displayed in FIG. 10A, and a record obtained following 10 minutes exposure to alternating magnetic field at frequency of 16 Hz intensity of 4 μT at the surface of the chest is displayed in FIG. 10B. A decrease in QRS amplitude following exposure to WMF of 16 Hz is exhibited.

In the tissue Doppler study, “E” and “A” wave's area and amplitude were measured. No changes were observed during and following diastolic function at transmitral, septal and lateral views. “E” wave was 76.9 cm/sec in controls and 76.7 cm/sec during application of alternating magnetic field while “A” wave was 63.2 cm/sec in controls and 63 for cm/sec (transmitral).

FIG. 11 shows an example of a tissue Doppler study. No changes were observed before and during the application of alternating magnetic field in the diastolic function in humans with healthy hearts. This does not necessarily attest to possible changes that may occur in patients with ailing hearts such as those suffering from congestive heart failure who are critically dependent for maintaining a minimally adequate cardiac function dependent on their reserves of intracellular Ca2+ stores.

### Discussion

In both the in vivo and in vitro experiments (see Example 1 above) the observed changes induce by the alternating magnetic field of the present embodiments, started 5-10 min following initial exposure. Such period is reasonable for a biologic tissue to respond to an external stimulation. The conclusive results in cardiac experimental animals, isolated myocytes, and healthy human volunteers, clinch the notion that exposure of myocardial cells to the alternating magnetic field of appropriate frequency induces activation of sarcolemma K_{ATP} channels.

In isolated myocytes in culture the activation of K_{ATP} channels by a 16 Hz alternating magnetic field depleted the myocytes of intracellular Ca²⁺. Exposed healthy human volunteers, without previous history of heart failure and with normal left ventricular (LV) function as depicted in the ECG and echocardiography and by tissue Doppler demonstrated no appearance of any symptoms, did not exhibit any side effects. No arrhythmias, changes in heart rate or echocardiographic changes were observed. Yet there was a clear evidence of significant reduction in the amplitude of the QRS complex during exposure to the fields that lasted long after the field was arrested.

The results of the above experiments demonstrate that the alternating magnetic field of the present embodiments affects the electrophysiologic properties of myocardial cells in vitro and in vivo, and in healthy persons. Reduction of the amplitude of the QRS in the ECG was also observed in the rats, in vivo. The rats also exhibited shortening of the QT interval in the ECG following activation of K_{ATP} channels.

Reduction in the amplitude of the QRS complex in the ECG following exposure to a 16 Hz alternating magnetic field was a novel observation and an unexpected finding in two in vivo studies (rats as well as humans). Such changes evolved in all in vivo experiments about 10 min following initiation of the field and, recovery necessitated about 20-60 minutes.

Without being bound to any theory it is assumed that the reduction in QRS amplitude following the exposure to the alternating magnetic field is attributed to the hyperpolarization of the sarcolemma induced by the activation of potassium ATP channels. Hyperpolarization of the interior of the cell means that the cell becomes more negative compared to its previous normal level. During phase zero depolarization, the influx of sodium ions possessing positive charges, is counteracted by the enhanced negativity of the intra-cellular volume induced by reduced positively charged potassium and calcium ions. When phase zero depolarization in the action potential has an amputated amplitude, it is manifested in the surface ECG as an attenuation of the amplitude of the QRS complex.

Following are other explanations to the phenomenon:

1) Partial inactivation of the rapid sodium current (I_{Na}) during phase zero of the action potential occurring at the beginning of the action potential. This may manifest in the ECG as QRS complex amputation following the exposure to the alternating magnetic field.

2) In human hearts exposed to 16 Hz magnetic field, a single coil was placed over the chest of healthy individuals, usually at the location at the region of leads V3-V5, and in those particular leads the ECG, showed maximal amputation of the peak of the QRS complex. The position of the coil may have resulted in directing its maximal intensity of the fields directed perpendicularly to the anterolateral or anteroapical regions of the myocard. The greater effect of the field was therefore directed at those regions while rendering only minimal effects to other myocardial regions. Since the external ECG registers the vectorial summation of all electrical impulses occurring within the heart at any given point in time, the balanced effect of regions having complete sodium current (INa) inactivation, and those with no inactivation at all may be responsible for the observed changes in the amplitude attenuation of the QRS complex. Cairns et al (2004), for example, suggested that inactivated sodium channels related to regional ischemia are responsible for action potential amplitude reduction, and that it was accompanied by reduced muscle contraction.

The ability of the alternating magnetic field of the present embodiments to activate myocardial K_{ATP} channels as was concluded from the above experiments can serve as a practical therapeutic tool for conserving heart tissue and tissues of other organs in the events of ischemic distress.

The above experiments demonstrated that the opening of sarcollemal K_{ATP} following the exposure to the alternating magnetic field mimics the process of ischemic preconditioning (IPC), or anesthetic preconditioning (APC). The effect was induced by the alternating magnetic field at the region of the heart. The present Inventors presented the utilization of novel tool and methods to confront myocardial ischemia, such as expressed in the clinical phenomenon of inter-mediate coronary syndrome that not infrequently leads to infarction, or become beneficial in the evens of anginal syndrome, or as a prior induction applied to the patient's heart before open heart surgery or PCI.

### Example 3

### The Effect of 50 Hz Magnetic Fields on Human Heart

### Methods

The present Inventors investigated evolving ECG changes in nine healthy volunteers who approached public transformers or power-lines for duration of 10 minutes. The volunteers (7 men and 2 women) had no known or apparent heart disease. The ECG signals were recorded continuously by Holter recorders and were analyzed at a later stage.

Following 10 minutes of exposure to a 50 Hz electromagnetic field of 7-15 microtesla, the volunteers experienced a 20-30 minutes recovery period away from the radiating sources.

### Results

In all volunteers, the QRS amplitude of the ECG was reduced at a range varying between 5.5% and 37.7%. The average reduction of the peak of the QRS complex was about 16.7%±9.67%. No symptoms or side effects were noted, except that few of the volunteers (3) described a tingling sensation in their fingers while standing in close proximity (20-30 cm) to the power source.

### Example 4

### Treatment of Diabetic Foot

Several diabetic subjects were treated by the alternating magnetic field of the present embodiments. For each subject, one foot was exposed to a 16 Hz magnetic field having intensity of 300 nT to 7 microtesla at the origin of the coil (see FIG. 1D). About 5-10 minutes following the exposure, the skin of both feet of the subject gained an increase in temperature. The increase ranged from about 0.6° C. to about 1.7° C. The subjects acquired superficial arterial pulses in both feet.

The bilateral effect involving both feet while only one was exposed to the WMF, resulted through a mechanism of entrainment promoted via the autonomous nervous system responsible for creating near equal effects to be demonstrated in both feet.

The working mechanism of the effect of the 16 Hz magnetic field on the foot is as described before. Activation of K_{ATP} channels residing in the arteriolar myocytes in the dermal layer of the foot skin, affected by the alternating magnetic field, promote an increase in dermal arteriolar blood perfusion, that enhance local skin blood flow, raise foot skin temp and in due time favors therapeutic results.

### Example 5

### Treatment of Facial Skin

The cheeks of 10 human volunteers were exposed to a 16 Hz magnetic field using the patch shown in FIG. 4A. The intensity of the alternating magnetic field was 100-300 nT at the emission site of the array of coils. The duration of exposure ranged between 10 minutes and 15 minutes.

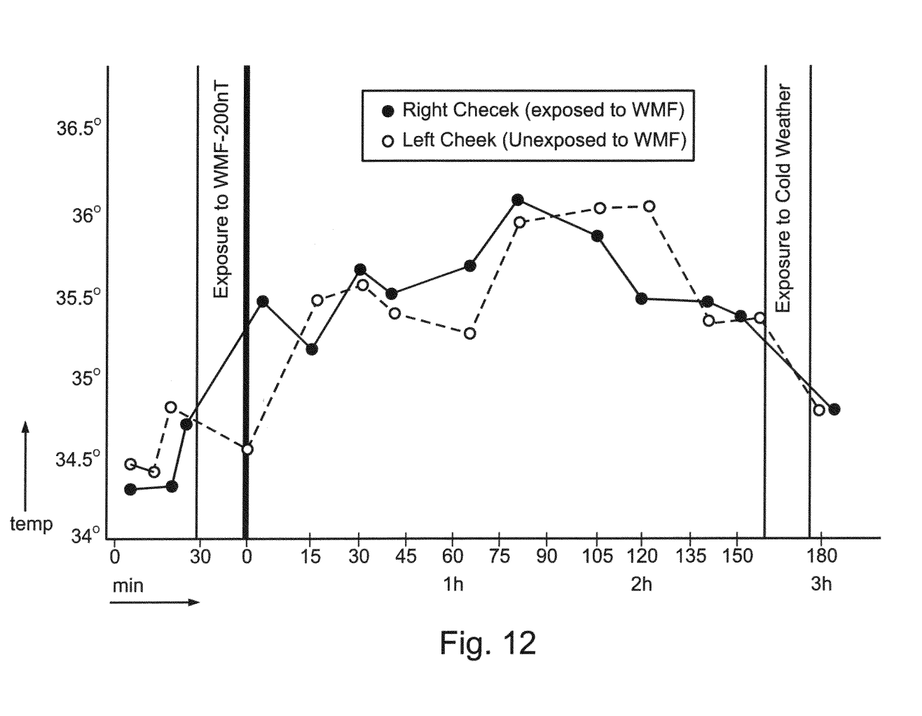

Following exposure to the alternating magnetic fields, the facial skin temperature was increased by 1.1-1.7° C. (see FIG. 12). Also, enhancement in the pinkish tint of the cheeks compared with previous control state was observed.

Such effects rendered to the cheeks are related to increased blood supply to the cheeks, due to arteriolar vasodilation within the dermal layer of the skin occurring in both cheeks despite the fact that only one cheek was exposed to the alternating magnetic field. On the average, the effect lasted about two hours following the termination of treatment.

### REFERENCES

Adair, R. K., R. D. Astumian, and J. C. Weaver. (1998). Detection of weak electric fields by sharks, rays, and skates, Chaos 8:576-587.
Adair, R. K. (2006). Comment: Analyses of models of ion actions under the combined action of AC and DC magnetic fields, Bioelectromagnetics 27(4): 332-334.
Albertini A., P. Zucchini, G. Noera, R. Cadossi, C. P. Napoleone, and A. Pierangeli. (1999). Protective effect of low frequency low energy pulsing electromagnetic fields on acute experimental myocardial infarcts in rats. Bioelectromagnetics 20:372-377
Alexander C M & Harrison P J. The bilateral reflex control of the trapezius muscle in humans. Exp Brain Res, (2002); 142(3):418.
Ali F. M. Mohamed M. R. (2003), Effect of 50 Hz, 0.2 mT magnetic fields on RBC Properties and heart functions of albino rats. Bioelectromagnetics 24(8), 535-45
Astumian, R. D., R. K. Adair, and J. C. Weaver. (1997). Stochastic resonance at the single-cell level. Nature 388:632-633.
Balian V. Galli M. Marcassa C. Cecchin G. Child M. Barlocco F. Petrucci E. Filippini G. Michi R. and Onofri M. (2006). Intracoronary ST-segment shift soon after elective percutaneous coronary intervention accurately predicts periprocedural myocardial injury, Circulation; 114: 1948-1954.
Bawin S., and Adey W. (1976). Sensitivity of calcium binding in cerebral tissue to weak environmental electric fields oscillating at low frequency. PNAS. 73(6):1999-2003.
Bawin S, L. Kaczmarek and, W. Adey. (1975). Effects of modulated VHF fields on the central nervous system. Ann. NY. Acad. Sci. 247: 74-81.
Bezanilla F. (2002). Voltage sensor movements. J. Gen. Physiol. 120:465-473.
Benzi R, Parisi G, Suterra A Valpiani A. Stochastic resonance in climatic changes. Tellus. 34, 10-16, (1982).
Blackman C. F., S. G. Benane, L. Kinney, W. Joines, and D. E. House. (1982). Effects of ELF fields on calcium-ion efflux from brain tissue in vitro. Radiat. Res. 92:510-520.
Blackman C. F., S. G. Benane, J. Rabinowitz, D. E. House, and W. Joines. (1985). A role of the magnetic field in the radiation induced efflux of calcium ions from brain tissue in vitro. Bioelectromagnetics 6:327-337.
Blanchard J. P. Blackman C. F. Clarification and application of an ion parametric resonance mode for magnetic field interaction with biological systems. Bioelectromagnetics, (1994), 15(3): 217.
Borjanovic S. S. Jankovic S. M. Pejovic Z. (2005), ECG changes in humans exposed to 50 Hz magnetic fields. J Occup Health. 47(5), 391-396
Cao C, Healey S, Amaral A, Lee-Couture A, Wan S, Kouttab N, Chu W, Wan Y, ATP-sensitive potassium channel: A novel target for protection against UV-induced human skin cell damage. (2007) J Cell Physiol, 212: 252-263.
Cohen M. V. Bains C. P. and Downwy J. M., Ischemic preconditioning: from adenosine receptor to K_{ATP} channel. Annual Review of Physiology, (2000), 62: 79.
Collins J J, Carson C and Imhoff T T. Stochastic resonance without tuning, Nature, 376: 236-238 (letter to Nature Jul. 25, 2000).
Collins J J, Imhoff T T, and Grigg P. Noise enhanced tactile sensation. (1996) Nature, 383: 770
Cui Y, Giblin P, Clapp L H and Tinker A. A mechanism for ATP-sensitive potassium channel diversity: Functional coassembly of two pore-forming subunits. PANS (2001), 98:729-734.
Davila, A. F., M. Winklhofer, V. P. Shcherbakov, and N. Petersen. (2005). Magnetic pulse affects putative magnetoreceptor mechanism. Biophys. J. 89: 56-63.
Del Giudice E. Fleishman M. Preparata G Talpo G, On the unreasonable effects of ELF magnetic fields upon a system of ions. Bioelectromagnetics, (2002), 23(7): 522
DiCarlo, A. L., J. M. Farrell, T. Litovitz. A, (1999). Myocardial protection conferred by electromagnetic fields. Circulation 99:813-816
Fadel M. A, S. M. Wael, R. M. Mostafa. (2003). Effect of 50 Hz, 0.2 mT magnetic fields on RBC properties and heart functions of albino rats, Bioelectromagnetics, 24: 535-545.
Fixler D, Tirosh R, Zinman T Shainberg A Deutch M, Differential aspects in ratio measurements of [Ca2+] I relaxation in cardiomyocyte contraction following various drug treatments, Cell Calcium 31 (2002), pp. 279-287.
Galvanovskis J, Sandblom J, Bergqvist B, Galt S, Hamnerius Y. (1999). Cytoplasmic Ca²⁺ oscillations in human leukemia T-cells are reduced by 50 Hz magnetic fields. Bioelectromagnetics 20:269-276.
Gammaitoni, L. Hänggi, P. Jung, P., and F. Marchesoni. (1998). Stochastic Resonance. Rev. Mod. Phys. 70, pp. 223-288.
Jauchem J R. Exposure to extremely-low frequency electromagnetic fields and radiofrequency radiation: Cardiovascular effects in humans. Int Arch Occup Environ Health, 70: 9-21 (1997).
Jeong, J. H., J. S. Kim, B. C. Lee, Y. S. Min, D. S. Kim, J. S. Ryu, K. S. Soh, K. M. Seo, U. D. Sohn. (2005). Influence of exposure to electromagnetic field on the cardiovascular system, Autonomic & Autacoid Pharmacology, 25:17-23.
Gumina R G, O′Cochlain D F, Kurtz E C, Bast P., Pucar D., Mishra P., Mild T., Seino S., Macura S., and Terzic A. K_{ATP} channel knockout worsen myocardial calcium stress load in vivo and impairs recovery in stunned heart. Am J Physiol Heart Circ Physiol 292: H1706-1713 (2006).
Gross G J. Peart J N. K_{ATP} channels and myocardial preconditioning: An update. A J Physiol, (2003); 285: 921
Hang C. L. Wang C. P. Yip H. K. et al, (2005), Early administration of intracoronary verapamil improves myocardial perfusion during PCI for acute myocardial infarction. Chest; 128: 2593.
Heusch P., Skyschally A., Leineweber K., Haude M., Erbel R., & Heusch G. The interaction of coronary microembolization and ischemic preconditioning: a third window of cardioprotection through TNF-alpha. (2007), Arch Med Sci 3, 2: 83-92.
Herrmann J. (2005). Peri-procedural myocardial injury: 2005 update, European Heart J, 26(23):2493-2519.
Johnsen, S., K. J. Lohmann. (2005). The physics and neurobiology of magnetoreception. Nature Reviews Neuroscience. 6, pp. 703-712.
Kalmijn, A. J. (1982) Electric and magnetic field detection in elasmobranch fishes, Science 218: 916-918.
Korpinen, L. and Partanen J. (1996). Influence of 50-Hz electric and magnetic fields on human blood pressure, Radiat Environ Biophys. 35(3):199-204.
Lafferty K., De Trafford J C., Potter C., Roberts V C., Cotton L T. (1985), Reflex vascular responses in the finger to contra lateral thermal stimuli during the normal menstrual cycle: a hormonal basis to Raynaud's phenomenon? Clin Sci (Lond). 68(6): 639-45
Liboff A. R., S. D. Smith, B. R. McLeod. (1987). Experimental evidence for ion cyclotron resonance mediation of membrane transport. In: Blank M, Findl E, editors, Mechanical Approaches to Interactions of Electric and Electromagnetic Fields with Living Systems, Plenum Press, NY, pp. 281-296.
Liboff, A. R. (1997). Electric-Field Ion Cyclotron Resonance, Bioelectromagnetics 18: 85-87.
Malin S R C & Strivastava B J, Correlation between heart attacks and magnetic activity, Nature, (1979); 277, 646-648.
R. Mazhari, H. B. Nuss, A. A. Armoundas, R. L. Winslow, E. Marban. (2002). Ectopic expression of KCNE3 accelerates cardiac repolarization and abbreviates the QT interval, J. Clin. Invest. 109:1083-1090.
McLeod, B. R., A. R. Liboff. (1986). Dynamic characteristics of membrane ions in multifield configurations of low-frequency electromagnetic radiation, Bioelectromagnetics 7 pp. 177-189.
Menconi, M. C. M. Pellegrini, M. Pellegrino, D. Petracchi. (1998). Periodic forcing of a single ion channel: dynamic aspects of the open-closed switching, Eur. Biophys. J. 27, pp. 299-304.
Moore R L, Myocardial K_{ATP} channels are critical to Ca2+ homeostasis in the metabolically stressed heart in vivo. Am J Physiol Heart Circ Physiol, 292(4): H1692-H1693. (2007).
Michaels A. D. Appleby M. Often M. H. Dauterman K. Ports T. A. Chou T. M. and Gibson C. M. Pretreatment with intragraft verapamil prior to percutaneous coronary intervention of saphenous vein graft lesions: results of the randomized, controlled vasodilator prevention on no-reflow (VAPOR) trial. (2002) J Invasive Cardiol. 14(6): 299-302.
Nichols C G, K_{ATP} channels as molecular sensors of cellular metabolism. Nature, (2006), 40: 470-476.
Niehuis M. B. Ottervanger J. P. Dambrink J-H. E. Dikkeschei L. D. Suryapranata H. Hoomtje J. C. A. de Boer M. J. Gosselink A. T. M. and Zijlstra F., (2007) Troponin T elevation and prognosis after multivessel compared with single percutaneous coronary intervention. Neth Heart J. 15(5): 178-183.
Okada T, Otani H, Wu Y, Uchiyama T, Kyoi S, Hattori R, Sumida T<F IJIARA H, Imamura H. Integrated pharmacological preconditioning and memory of cardioprotection: role of protein kinase C and phosphatidylinositol 3-kinase. Am J Physiol Heart Circ Physiol, (2005), 289:H761-H767.
Pazur A. (2004). Characterization of weak magnetic field effects in an aqueous glutamic acid solution by nonlinear dielectric spectroscopy and voltammetry, BioMagnetic Research and Technology 2(8) pp. 1-11.
Petracchi, D., M. Pellegrini, M. Pellegrino, and F. Moss. (1994). Periodic forcing of K⁺ channel at various temperatures. Biophys. J. 66, pp. 1844-1852
Prasad A. Singh M. Lerman A. Ryan J. Lennon M. S. Holmes D. R. and Rihal C. S., (2006). Isolated elevation in Troponin T after percutaneous coronary intervention is associated with higher long-term mortality, J. Am. coll. Card; 48:1765-1770.
Ritz, T., P. Thalau, J. Philips, R. Wiltschko, and W. Wiltschko. (2004). Resonance effects indicate a radical-pair mechanism for avian magnetic compass. Nature 429: 177-180.
Sait, M. L, Wood, A. W and Sadafi, H. A. (1999). A study of heart rate And heart rate variability in human subjects exposed to occupational levels of 50 Hz circularly polarized magnetic fields, Med Eng. Phys 21:361-369.
Savitz D. A, D. Liao, A. Sastre, R. C. Kleckner and R. Kavet. (1999). Magnetic Field Exposure and Cardiovascular Disease Mortality among Electric Utility Workers. Am. J. of Epidemiol. 149(2): 135-142.
Shneyvays, V., L. Mamedova, T. Zinman, K. Jacobson, A. Shainberg. (2001), Activation of A₃ adenosine receptor protects against doxorubicin-induced cardiotoxicity, J Mol Cell Cardiol. 33, pp. 1249-1261.
Schwartz J L, House D E, Mealing G A. Exposure of frog hearts to CW or amplitude modulated VHF field: selective efflux of calcium ions at 16 Hz. Bioelectromagnetics 11 (4), 349-358, (1990).
Schwartz J R, Jeoffrey A, Mealing G A, Calcium-ions movement and contractility of atrial strips of frog heart are not affected by low-frequency-modulated 1 GHz electromagnetic radiation. Bioelectromagnetics; 14: 6 521-533 (1993).
Sigg, D. and F. Bezanilla. (2003). A Physical Model of Potassium Channel Activation: From Energy Landscape to Gating Kinetics, Biophys. J. 84, pp. 3703-3716.
Sigg, D., F. Bezanilla, and E. Stefani. (2003). Fast gating in the Shaker K+ channel and the energy landscape of activation, PNAS 100, pp. 7611-7615.
Sigg, D., H. Qian, F. Bezanilla. (1999). Kramers' diffusion theory applied to gating kinetics of voltage-dependent ion channels, Biophys. J. 76, pp. 783-803.
Smith, S. (1987). Calcium cyclotron resonance and diatom mobility, Bioelectromagnetics 8 pp. 215-227.
Stadnicka A, Marinovic J, Bienegreaber M, Bosnjak Z J. (2006) Impact in vivo preconditioning by isoflurane on ATP sensitive potassium channels in the rat heart: lasting modulation of nucleotide sensitivity during early memory period. Anesthesiology 104: 503-510.
Sun H S., Feng Z P., Miki S., and French R J. (2006) Enhanced neuronal damage after ischemic insults in mice lacking Kir6.2 containing ATP-sensitive K+ channels. J Neurophysiol 95: 2590-2601.
Turner L A, Fujimoto K, Stadnicka A, Bosnjak Z J, Kwok W M. The interaction of isoflurane and protein kinase C-activators on sarcolemmal K_{ATP} channels. Anesth Analg (2005); 100: 1680-1686.
Ward I M., Neiman A., & Moss F. (2002). Stochastic resonance in psychophysics and in animal behavior. Biological Cybernetics, 87: 91
Wang Y. Ahmad N, Wang B, and Ashraf M. Chronic preconditioning: a novel approach for cardiac protection. Am J Physiol Circ Physiol 292: H2300-H2305 (2007).
Weaver, J. C., T. E. Vaughan, R. D. Astumian. (2000). Biological sensing of small field differences by magnetically sensitive chemical reactions. Nature 405, pp. 707-709.
Wiesenfeld K., & Moss F. Stochastic resonance and the benefits of noise: from ice ages to crayfish and SQUIDs. Nature; (1995), 373: 6509.
Wolke S, Neibig U, Elsner R, Gollnick F, Meyer R. Calcium homeostasis of isolated heart muscle cells exposed to pulsed high-frequency electromagnetic fields. Bioelectromagnetics, (1996); 17: 144-153.
Yao Z, Mizumura T, Mei D A, Gross G J. K_{ATP} channels and memory of ischemic preconditioning in dogs: synergism between adenosine and K_{ATP} channels. Am J Physiol (1997); 272H334-H342.
Yellen, G. (2002). The voltage-gated potassium channels and their relatives, Nature 419, pp. 35-42.
Zeng, J., K. R. Laurita, D. S. Rosenbaum, Y. Rudy. (1995), “Two components of the delayed rectifier K+ current in ventricular myocytes of the Guinea pig type, Circ. Res. 77, pp. 140-152.
Zhadin, M. N. Barnes F. (2005). Frequency and amplitude windows in the combined action of DC and low frequency AC magnetic fields on ion thermal motion in a macromolecule: Theoretical analysis. Bioelectromagnetics 26:323-330.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of preconditioning a potentially ischemic myocardium of a subject, comprising exposing the myocardium to an alternating magnetic field at a frequency of from 15.5N Hz to about 16.5N Hz or from 7.8 Hz to 8.2 Hz and duration selected so as to activate potassium ATP channels in the myocardium, wherein N is a positive integer.

2. A method of treating a myocardium under an ischemic distress event of a subject, comprising exposing the myocardium to an alternating magnetic field at a frequency of from 15.5N Hz to about 16.5N Hz or from 7.8 Hz to 8.2 Hz and duration selected so as to activate potassium ATP channels in the myocardium, wherein N is a positive integer.

3. The method of claim 2, wherein said ischemic distress event comprises at least one event selected from the group consisting of angina, unstable angina, intermediate coronary syndrome, myocardial infarction, and ischemia due to no-reflow during percutaneous coronary intervention.

4. The method of claim 1, further comprising performing a percutaneous coronary intervention following said exposure to said alternating magnetic field.

5. The method of claim 4, wherein said exposure to said alternating magnetic field is performed so as to reduce or prevent no-reflow phenomenon in said myocardium.

6. The method of claim 1, further comprising monitoring ECG signals from the subject.

7. The method of claim 6, wherein said exposure to said alternating magnetic field is at least until a QT interval of characterizing said ECG signal is shortened. by at least 5%.

8. A method of treating a smooth muscle conduit of a subject, comprising exposing the smooth muscle conduit to an alternating magnetic field at a frequency of from 15.5N Hz to about 16.5N Hz or from 7.8 Hz to 8.2 Hz and duration selected so as to activate potassium ATP channels in cells of the smooth muscle conduit, wherein N is a positive integer.

9. The method of claim 8, wherein said smooth muscle conduit comprises a blood vessel.

10. The method of claim 8, wherein said smooth muscle conduit comprises a retinal arteriole.

11. The method of claim 8, wherein said smooth muscle conduit comprises a gastrointestinal tract.

12. The method of claim 8, wherein said smooth muscle conduit comprises a bladder.

13. The method of claim 8, wherein said smooth muscle conduit comprises a kidney.

14. The method of claim 8, wherein said smooth muscle conduit comprises a trachea.

15. A method enhancing blood perfusion of a dermal layer of a subject, comprising exposing the dermal layer to an alternating magnetic field at a frequency of from 15.5N Hz to about 16.5N Hz or from 7.8 Hz to 8.2 Hz and duration selected so as to activate potassium ATP channels in the dermal layer, wherein N is a positive integer.

16. The method of claim 15, wherein said dermal layer is a part of a face of the subject.

17. The method of claim 15, wherein said dermal layer is a part of a scalp of the subject.

18. The method of claim 1, further comprising administrating to the subject a potassium ATP channel opener drug.

19. The method of claim 15, wherein the subject is diabetic.

20. A drug delivery method, comprising administrating to a subject a therapeutic amount of at least one drug and exposing an organ of said subject to an alternating magnetic field at a frequency of from 15.5N Hz to about 16.5N Hz or from 7.8 Hz to 8.2 Hz and duration selected so as to activate potassium ATP channels in said organ, wherein N is a positive integer.

21. A method of reducing the likelihood for skin damage to an organ of a subject being irradiated by solar radiation, comprising exposing the organ to an alternating magnetic field at a frequency of from 15.5N Hz to about 16.5N Hz or from 7.8 Hz to 8.2 Hz while the organ is irradiated by the solar radiation.

22. The method of claim 1, wherein said exposure to said alternating magnetic field comprises applying said alternating magnetic field to a skin of the subject.

23. A medical device, comprising a patch designed attachable to a skin of a subject, and an alternating magnetic field generator mounted on or integrated in said patch and configured for generating an alternating magnetic field in the direction of said skin.

24. The device of claim 23, wherein said patch comprises at least one medicament incorporated therein for intradermal or transdermal delivery of said at least one medicament to the subject.

25. A mobile system, comprising a cellular telephone unit and an alternating magnetic field generator configured for generating an alternating magnetic field at a frequency of from 15.5N Hz to about 16.5N Hz or from 7.8 Hz to 8.2 Hz, wherein N is a positive integer.

26. The system of claim 25, wherein said alternating magnetic field generator is detachable from said cellular telephone unit.

27. The device of claim 23, further comprising a user interface for selecting or adjusting at least one parameter characterizing said alternating magnetic field.

28. The method of claim 1, wherein said alternating magnetic field is generated at an intensity which is less than 10 microteslas.

29. A shielding device, comprising a ferromagnetic core having a closed shape and being embedded in a garment designed to be worn by a subject and cover an organ thereof, wherein said ferromagnetic core is designed and constructed to substantially prevent penetration of an alternating magnetic field at a frequency of less than 300 Hz therethrough.

30. The device of claim 29, wherein said organ is a chest.

31. The device of claim 29, wherein said organ is a abdomen.

32. The device of claim 31, the subject is a pregnant woman.

33. The device of claim 29, wherein said ferromagnetic core comprises a plurality of layers.
